(19) United States
(12) Patent Application Publication
(10) Pub. No.: US 2024/0425934 A1
(43) Pub. Date: Dec. 26, 2024

(54) METHODS OF SELECTING AND TREATING CANCER SUBJECTS THAT ARE CANDIDATES FOR TREATMENT USING INHIBITORS OF PARP

(71) Applicant: Arima Genomics, Inc., Carlsbad, CA (US)

(72) Inventors: Anthony Schmitt, Apex, NC (US); Kristin Sikkink, Fallbrook, CA (US)

(73) Assignee: Arima Genomics, Inc., Carlsbad, CA (US)

(21) Appl. No.: 18/825,852

(22) Filed: Sep. 5, 2024

Related U.S. Application Data
(63) Continuation of application No. PCT/US2023/063806, filed on Mar. 6, 2023.
(60) Provisional application No. 63/418,416, filed on Oct. 21, 2022.
(60) Provisional application No. 63/400,869, filed on Aug. 25, 2022.
(60) Provisional application No. 63/400,865, filed on Aug. 25, 2022.
(60) Provisional application No. 63/322,745, filed on Mar. 23, 2022.
(60) Provisional application No. 63/322,748, filed on Mar. 23, 2022.

(51) Int. Cl. C12Q 1/6886; A61K 45/06

(52) U.S. Cl. CPC C12Q 1/6886; A61K 45/06; C12Q 2600/106; C12Q 2600/156

(57) ABSTRACT

The technology relates in part to methods of selecting for and/or treating subjects having cancer, where the subjects are identified as having at least one genetic structural variant that renders them suitable candidates for a treatment method that includes the administration of at least one homologous recombination deficient direct therapy, such as an inhibitor of a polyadenosine diphosphate-ribose polymerase (PARP) enzyme.

### CROSS REFERENCE TO RELATED APPLICATIONS

This application claims the benefit under 35 U.S.C. § 119(e) of U.S. provisional application No. 63/322,745, filed Mar. 23, 2022, U.S. provisional application No. 63/400,865, filed Aug. 25, 2022, U.S. provisional application No. 63/322,748, filed Mar. 23, 2022, U.S. provisional application No. 63/400,869, filed Aug. 25, 2022, and U.S. provisional application No. 63/418,416, filed Oct. 21, 2022. The entire contents of each of these referenced applications are incorporated by reference herein.

### FIELD

The technology relates in part to methods of selecting for and/or treating subjects having cancer, where the subjects are identified as having at least one genetic structural variant that renders them suitable candidates for a treatment method that includes the administration of homologous recombination deficient direct therapy, such as an inhibitor of a polyadenosine diphosphate-ribose polymerase (PARP) enzyme.

### BACKGROUND

Cancers are often caused by genetic alterations, which include mutations (e.g., point mutations) and structural variations (e.g., translocations, inversions, insertions, deletions, and duplications). Genetic alterations can prevent certain genes from working properly. Genes that have mutations and/or structural variations that are linked to cancer may be referred to as cancer genes. Certain types of cancers have been linked to specific genetic alterations. However, there are cancers for which specific genetic alterations have not yet been identified.

A subject may acquire cancer-causing genetic alterations in a number of ways. In certain instances, a subject is born with a genetic alteration that is either inherited from a parent or arises during gestation. In certain instances, a subject is exposed to one or more factors that damage genetic material (e.g., UV light, cigarette smoke). In certain instances, genetic alterations arise as the subject ages. Given how cancer can cause cells to go haywire and replicate in an uncontrolled, invasive fashion, it is not unexpected that the same cancers often are caused by different genetic alterations, or by the synergistic effect of more than one genetic alteration.

Thus, the genetic make-up of subjects having cancer, even the same type of cancer, can vary widely, depending on differences in their genetic alterations. These differences in turn can lead to differences in their responsiveness to treatments. While the standard of care treatment may be effective in a majority of subjects having a particular type of cancer, e.g., triple-negative breast cancer or glioblastoma multiforme, certain subjects are not responsive or are less responsive to the standard of care treatment. For such subjects, there is a need to identify alternate treatments that can be administered in addition to, or instead of, the standard of care treatment. Provided herein are methods of selecting and/or treating cancer subjects (patients) that are suitable candidates for alternate treatment regimens based on the presence of one or more genetic alterations that render them responsive to an alternate treatment regimen.

Small variants, such as point mutations and InDels, are the mutation types known to compromise the function of homologous recombination repair (HRR) genes in cancer cells, rending them homologous recombination deficient (HRD). Described herein are fusions (proximity or gene), a new type of mutation compromising the function of HRR in cancer cells. If a fusion (based on any method, such as but not limited to spatial-proximal contiguity analysis) is identified associated with a HRR gene, patients will respond better to therapies that are known to be efficacious in the context of homologous recombination deficiency (HRD), including but not limited to PARP inhibitors or platinum-based therapies (Lise M. van Wijk, Andreea B. Nilas, Harry Vrieling & Maaike P. G. Vreeswijk (2022) RAD51 as a functional biomarker for homologous recombination deficiency in cancer: a promising addition to the HRD toolbox?, Expert Review of Molecular Diagnostics, 22:2, 185-199, DOI: 10.1080/14737159.2022.2020102).

Applicants have discovered that HRR gene rearrangements impact several HRR genes (see Table 1 and Table 8) and are found in several cancer types (See Table 8), indicating that HRR gene rearrangements may be a “pan-cancer” biomarker for HRD and indicate which patients will respond favorably to HRD directed therapies. The data also indicate that the prevalence of HRR alterations is higher than currently known, given that rearrangements of HRR genes was an alteration type not previously considered to alter function of HRR genes. Evidence that HRR gene rearrangements lead to HRD can be observed by the degree of genomic instability in a sample, measured in the nucleic acid analyses described herein as the total number of genomic rearrangements in a sample, with a higher number of rearrangements indicative of HRD. Using the spatial-proximal contiguity analyses described herein, a common feature of samples with HRR gene rearrangements is a higher total number of genomic rearrangements than samples without HRR gene rearrangements, indicating HRD. To illustrate this point using matched analyses of the same tumor type with and without HRR gene alterations, we show significantly more intra- and inter-chromosomal rearrangements in samples with HRR gene rearrangements compared to those without (See FIGS. 2-5). It is therefore anticipated that detection of HRR gene rearrangements, such as by using a spatial proximal contiguity analysis, may be indicative of HRD correlates with response to HRD directed therapies.

In addition, it is anticipated that there are gene-agnostic, overall signals from a spatial proximal contiguity analysis (Hi-C) that would be indicative of HRD and predict response to HRD-directed therapies. For example, even in the absence of detecting a gene rearrangement involving a known HRR gene, the total number of rearrangements detected could be indicative of HRD, with a higher number of fusions being indicative of HRD, or indicative of an increased likelihood of HRD and therefore predictive of response to therapies that are known to be efficacious in the context of HRD, including but not limited to PARP inhibitors or platinum-based therapies. Rearrangement-based HRD detection, such as by using a spatial proximal contiguity analysis, could be carried out using genome-wide spatial proximal contiguity analysis (Hi-C), or in the context of targeted assays such as targeted spatial proximal contiguity analysis (Capture-HiC or other targeted techniques).

It is expected that the total number of fusions found in a multitude of tumor types, including especially sarcomas, will increase when there is a disruption (such as, but not limited to a structural variant breakpoint) near or in an HRR gene.

### SUMMARY

Provided in certain aspects are methods of treating a subject that has, or is suspected of having, cancer, where the methods include: a) identifying and/or selecting a subject comprising a structural variant in the genome of the subject, where the location of the structural variant, or a breakpoint of the structural variant, is associated with and/or adjacent to any one or any combination of the homologous recombination repair (HRR) genes shown in Table 1; and b) if the subject has cancer, treating the subject so identified and/or selected with a treatment that inhibits a polyadenosine diphosphate-ribose polymerase (PARP).

Also provided herein are methods of selecting a subject having cancer for treatment with an agent that inhibits a polyadenosine diphosphate-ribose polymerase (PARP), where the methods include: a) determining whether the subject comprises a structural variant in the genome of the subject, where the location of the structural variant, or a breakpoint of the structural variant, is associated with and/or adjacent to any one or any combination of the homologous recombination repair (HRR) genes shown in Table 1; and b) if the structural variant, or a breakpoint of the structural variant, is identified in a), selecting the subject for treatment with an agent that inhibits a polyadenosine diphosphate-ribose polymerase (PARP).

Also provided herein are methods of screening a subject having cancer for potential responsiveness to treatment with an agent that inhibits a polyadenosine diphosphate-ribose polymerase (PARP), where the methods include: a) determining whether the subject comprises a structural variant in the genome of the subject, where the location of the structural variant, or a breakpoint of the structural variant, is associated with and/or adjacent to any one or any combination of the homologous recombination repair (HRR) genes shown in Table 1; and b) if the structural variant, or a breakpoint of the structural variant, is identified in a), identifying the subject as a candidate for, and/or as potentially responsive to, treatment with an agent that inhibits a polyadenosine diphosphate-ribose polymerase (PARP).

In certain aspects of any of the methods provided herein, a breakpoint of the structural variant maps to a location between positions as shown in row 5 of Table for any one of the homologous recombination repair (HRR) genes shown in Table 1, where the positions are in reference to an HG38 human reference genome. In aspects, a breakpoint of the structural variant maps to a location between positions as shown in Row 6 of Table 8 for any one of the homologous recombination repair (HRR) genes shown in Table 1, where the positions are in reference to an HG38 human reference genome.

In certain aspects of any of the methods provided herein, the adjacent location of the structural variant and/or a breakpoint of the structural variant, is at a distance of about x base pairs from any one of the homologous recombination repair (HRR) genes shown in Row V of Table X, where x is the value shown in Row A or Row W (Closest distance to gene body) (bp) from the corresponding HRR gene, where the distance is measured from the 3′ end or 5′ end of the respective HRR gene, as indicated in Row T of Table X. In aspects, the adjacent location of the structural variant and/or a breakpoint of the structural variant is at a distance of about x base pairs to about x+5,000 bp from 3′ end or 5′ end of the respective HRR gene, where x is the value shown in Row A or Row W (Closest distance to gene body) (bp) from the corresponding HRR gene and where the distance is measured from the 3′ end or 5′ end of the respective HRR gene, as indicated in Row T of Table X. In aspects, the distance is a linear distance.

In certain aspects of any of the methods provided herein, the structural variant and/or a breakpoint of the structural variant includes an ectopic portion of genomic DNA from positions selected from the group consisting of: positions found in row 5, row 6, row 22, row 23 of Table 8.

Also provided herein are methods for treating a cancer that include administering a polyadenosine diphosphate-ribose polymerase (PARP) enzyme inhibitor to a subject in need thereof in an amount effective for treating the cancer. Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like. In some embodiments, a cancer is a rare cancer. In some embodiments, a cancer is glioma. In some embodiments, a cancer is glioblastoma. In some embodiments, a cancer is pediatric glioblastoma. In some embodiments, a cancer is glioblastoma multiforme/anaplastic astrocytoma with piloid features (ANA PA). In some embodiments, a cancer is a sarcoma. In some embodiments, a cancer is uterine cancer. In some embodiments, a cancer is uterine leiomyosarcoma. In some embodiments, a cancer is uterine myxoid leiomyosarcoma. In some embodiments, a cancer is metastatic high-grade sarcoma, uterine origin. In some embodiments, a cancer is a brain tumor. In some embodiments, a cancer is a benign brain tumor. In some embodiments, a cancer is an astrocytic brain tumor. In some embodiments, a cancer is subependymal giant cell astrocytoma (SEGA). In some embodiments, a cancer is pleomorphic xanthoastrocytoma (PXA). In some embodiments, a cancer is a malignant brain tumor. In some embodiments, a cancer is a bone cancer. In some embodiments, a cancer is chordoma. In some embodiments, a cancer is a central nervous system (CNS) tumor. In some embodiments, a cancer is meningioma. In some embodiments, a cancer is an embryonal tumor. In some embodiments, a cancer is an embryonal central nervous system tumor. In some embodiments, a cancer is embryonal tumors with multilayered rosettes (ETMR). In some embodiments, a cancer is a kidney/renal cancer. In some embodiments, a cancer is a primitive neuroectodermal tumor (PNET). In some embodiments, a cancer is a kidney primitive neuroectodermal tumor (PNET). In certain embodiments, the cancer is any one of the types listed in Row 3 of Table 8.

In certain aspects of any of the methods provided herein, the subject is refractory to a standard of care treatment for cancer. In aspects, the standard of care treatment can include performing and/or administering one or more of the following: surgical tumor resection, radiotherapy, positron emission tomography (PET)-guided radiotherapy, positron emission tomography (PET)-guided dose escalated radiotherapy, laser interstitial thermal therapy, stereotactic radiosurgery (SRS), hypofractionated stereotactic radiotherapy (HFSRT), Tumor-Treating Fields (TTFields), chemoradiotherapy, brachytherapy, carmustine implantable wafers, temozolomide, vincristine, interferon, bevacizumab, onartuzumab, a nitrosourea, procarbazine, enzastaurin, teniposide, cytarabine, vincristine, irinotecan, carboplatin, dasatinib, temsirolimus, erlotinib, sorafenib, veliparib, galunisertib, cediranib, vorinostat, panobinostat, dianhydrogalactitol (VAL-083) and paclitaxel poliglumex (PPX).

In certain aspects of any of the methods provided herein, the treatment that inhibits a PARP enzyme, which is administered in addition to or instead of the standard of care treatment, is a treatment comprising administering a medicament selected from the groups consisting of: olaparib (Lynparza), niraparib, (Zejula), rucaparib (Rubraca), and talazoparib (Talzenna).

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1A shows a schematic of Capture-HiC data using target enrichment probes targeted to cancer genes in order to identify a structural variant (SV) that results in a gene fusion. FIG. 1B shows a schematic of Capture-HiC data using target enrichment probes targeted to cancer genes in order to identify an SV that results in a breakpoint outside of the targeted gene body.

FIG. 2A shows a genome-wide Hi-C analysis and heatmap of spatial proximal interactions between all chromosomes in the genome, and numerous inter-chromosomal rearrangements between different chromosomes in a driver-negative colorectal carcinoma tumor. This tumor had a detected HRR gene rearrangement involving the HRR gene BRIP1, and 23 total rearrangements in the sample. FIG. 2B shows a genome-wide Hi-C analysis and heatmap of spatial proximal interactions between all chromosomes in the genome in a driver-negative colorectal carcinoma tumor. This tumor did not have a detected HRR gene, and 2 total rearrangements in the sample.

FIG. 3A shows a genome-wide Hi-C analysis and heatmap of spatial proximal interactions within chr1 and 12 intra-chromosomal rearrangements within chr1 in a driver-negative Chordoma tumor. This tumor had a detected HRR gene rearrangement involving the HRR gene RAD54L, and 54 total rearrangements in the sample. FIG. 3B shows a genome-wide Hi-C analysis and heatmap of spatial proximal interactions within chr1 and 0 intra-chromosomal rearrangements within chr1 in a driver-negative Chordoma tumor. This tumor did not have a detected HRR gene rearrangement, and 6 total rearrangements in the sample.

FIG. 4A shows a genome-wide Hi-C analysis and heatmap of spatial proximal interactions within chr5 and 125 intra-chromosomal rearrangements within chr5 in a driver-negative uterine sarcoma tumor. This tumor had a detected HRR gene rearrangement involving the HRR gene RAD51B, and 158 total rearrangements in the sample. FIG. 4B shows a genome-wide Hi-C analysis and heatmap of spatial proximal interactions within chr5 and 0 intra-chromosomal rearrangements within chr5 in a driver-negative uterine sarcoma tumor. This tumor did not have a detected HRR gene rearrangement, and 1 total rearrangement in the sample.

FIG. 5A shows a genome-wide Hi-C analysis and heatmap of spatial proximal interactions within chr10 and 5 intra-chromosomal rearrangements within chr10 in a driver-negative lymphoma tumor. This tumor had a detected HRR gene rearrangement involving the HRR gene RAD51C, and 28 total rearrangements in the sample. FIG. 5B shows a genome-wide Hi-C analysis and heatmap of spatial proximal interactions within chr10 and 0 intra-chromosomal rearrangements within chr10 in a driver-negative lymphoma tumor. This tumor did not have a detected HRR gene rearrangement, and 1 total rearrangement in the sample.

### DETAILED DESCRIPTION

Provided herein are methods of treating a subject that has, or is suspected of having, cancer, where the methods include identifying and/or selecting a subject comprising a structural variant in the genome of the subject, where the structural variant and/or a breakpoint of the structural variant is associated with and/or adjacent to any one of the known homologous recombination repair (HRR) genes as shown in Table 1.

TABLE 1
HRR GENES

ATM
ATR
BARD1
BRCA1
BRCA2
BRIP1
CDK12
CHEK1
CHEK2
FANCA
FANCD2
FANCL
NBN
PALB2
PARP3
RAD51B
RAD51C
RAD51D
RAD54L
RPA1
SEM1

Also provided herein are methods of treating a subject that has, or is suspected of having, cancer, where the methods include identifying and/or selecting a subject comprising a structural variant in the genome of the subject, where the structural variant and/or a breakpoint of the structural variant is associated with and/or adjacent to a homologous recombination repair (HRR) gene.

In aspects of the methods provided herein, the genome of the subject comprises a structural variant associated with and/or adjacent to one or more of the HRR genes of Table 1.

Also provided herein are methods of selecting a subject having cancer for treatment an homologous recombination deficient direct therapy, such as an inhibitor of a polyadenosine diphosphate-ribose polymerase (PARP) enzyme. Also provided herein are methods of screening a subject having cancer for treatment with, and/or potential responsiveness to treatment with, an homologous recombination deficient direct therapy, such as an inhibitor of a polyadenosine diphosphate-ribose polymerase (PARP) enzyme.

The term “associated with,” as used herein, means that two features are connected in some regard, i.e., there is a relationship between the two features. For example, reference to an oncogene (first feature) being “associated with” a cancer (second feature), as used herein, means that the oncogene (generally having some aberration e.g., mutated, deleted, overexpressed, under expressed, expressing a mutant form of a protein, etc., relative to the corresponding “normal” or wild-type form) is consistently identified as being present in subjects having the cancer. The oncogene could cause the cancer or could arise as a downstream effect of the cancer. Similarly, reference to a structural variant, or a breakpoint of a structural variant, being “associated with” a gene means that the structural variant, or the breakpoint of a structural variant, is within the gene, partially overlaps with the gene, or is adjacent to the gene.

The terms “adjacent, “proximal to,” or “outside of,” as used herein in reference to a structural variant being outside of or adjacent to a gene, such as the HRR genes listed in Table 1, generally means that a breakpoint of a structural variant is not within the gene. The structural variant can contain the gene, such as at inversion of the gene, an insertion of the gene, a duplication of the gene, or the like, or can contain a portion of the gene. In certain aspects, the structural variant may not include the HRR gene, i.e., the structural variant does not contain the HRR gene, insertion, inversion, duplication or any portion thereof.

Adjacent generally means that the breakpoint of the structural variant is at a position or an equivalent distance that is between about 0 base pairs to about 1 Mb from the 5′ end or the 3′ end of an HRR gene, such as about 0, 10,00, 20,000, 30,00, 40,000, 50,000, 50,000, 70,000, 80,000, 90,000, 100,000, 125,000, 150,000, 175,000, 200,000, 250,000, 300,000, 350,000, 400,000, 450,000, 500,000, 550,000, 600,000, 650,000, 700,000, 750,000, 800,000, 850,000, 900,000, 950,000, 975,000, 999,999 or more base pairs. In certain embodiments, the breakpoint of the structural variant is at a position or an equivalent distance that is between about 0 base pairs to about 800,000 base pairs from the 5′ end or the 3′ end of an HRR gene, such as about 0, 100, 200, 300, 400, 500, 600, 700, 800, 900, 1000, 1500, 2000, 2500, 3000, 3500, 4000, 4500, 5000, 5500, 6000, 6,500, 7.000, 7,500, 8,000, 8,500, 9,000, 9,500 or 10,000 or more base pairs to about 50,000, 60,000, 70,000, 80,000, 90,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725.000, 750,000, 775,000 or 800,000 or more base pairs.

In some embodiments, a breakpoint of a structural variant maps to a particular location within a range of positions on a particular chromosome. In some embodiments, a breakpoint (e.g., receiving site) of a structural variant (e.g., insertion, translocation) maps to a particular location within a range of positions on a particular chromosome. In some embodiments, a breakpoint of a structural variant maps to a location between positions selected from the group consisting of: a position in Row 5 Table 8. In some embodiments, a breakpoint of a structural variant maps to a location between positions selected from the group consisting of: a position in Row 6 Table 8.

In some embodiments, a breakpoint (e.g., donor site) of a structural variant (e.g., insertion, translocation) maps to a particular location within a range of positions on a particular chromosome. A breakpoint for a donor site may map to a particular location within a range of positions that is different from the location of a receiving site. A breakpoint for a donor site may map to a particular location that is on the same chromosome as a receiving site or may map to a particular location that is on a different chromosome than a receiving site. In some embodiments, a breakpoint of a structural variant maps to a location between positions selected from the group consisting of: a position in Row 22 Table 8. In some embodiments, a breakpoint of a structural variant maps to a location between positions selected from the group consisting of: a position in Row 23 Table 8.

A structural variant may be defined in terms of a receiving site and a donor site. A receiving site may be referred to as a first partner or “partner 1” and a donor site may be referred to as a second partner or “partner 2.” In some embodiments, a structural variant may be defined in terms of comprising an ectopic portion of genomic DNA (i.e., a portion of genomic DNA at a receiving site from a different region of a chromosome or from a different chromosome). The ectopic portion may be referred to as a donor portion.

In some embodiments, a receiving site of a structural variant maps to a location between positions selected from the group consisting of: a position in Row 22 Table 8. In some embodiments, a receiving site of a structural variant maps to a location between positions selected from the group consisting of: a position in Row 23 Table 8. In some embodiments, a receiving site of a structural variant maps to a location between positions selected from the group consisting of: a position in Row 5 Table 8. In some embodiments, a receiving site of a structural variant maps to a location between positions selected from the group consisting of: a position in Row 6 Table 8.

In some embodiments, a structural variant may comprise an ectopic portion of genomic DNA (i.e., a portion of genomic DNA at a receiving site from a different region of a chromosome or from a different chromosome). The ectopic portion may be referred to as a donor portion. If the ectopic portion (donor portion) is from the same chromosome as the structural variant, the ectopic portion may be from a location outside of the position ranges provided above for certain structural variants. The ectopic portion may comprise genomic DNA from a genomic coordinate window provided herein, or part thereof. The ectopic portion may comprise genomic DNA from a genomic coordinate window provided herein, or part thereof, and may further comprise genomic DNA from a region outside of a genomic coordinate window provided herein.

In some embodiments, an ectopic portion of genomic DNA is characterized by its location (e.g., observed location for a given sample or samples) at a receiving site (e.g., at a structural variant site). In some embodiments, an ectopic portion is characterized by its location (e.g., observed location for a given sample samples) relative to the gene body of a gene and/or cancer gene. A gene body of a gene and/or cancer gene generally refers to a part of the gene and/or cancer gene that is transcribed. In some embodiments, an ectopic portion is within the gene body of a gene and/or cancer gene. In some embodiments, an ectopic portion is not within a gene body of a gene and/or cancer gene. For example, an ectopic portion may be located in an intronic region, intergenic region adjacent to a cancer gene, or within another gene adjacent to a cancer gene. In some embodiments, an ectopic portion is located at a position in proximity to the gene body for a gene and/or cancer gene. The term “in proximity” may refer to spatial proximity and/or linear proximity.

Spatial proximity generally refers to 3-dimensional chromatin proximity, which may be assessed according to a method that preserves spatial-proximal relationships, such as a method described herein or any suitable method known in the art. An ectopic portion may be located at a position in spatial proximity to the gene body for a gene and/or cancer gene when an ectopic portion and a gene and/or cancer gene (or a fragment thereof) are ligated in a proximity ligation assay or are bound by a common solid phase in a solid substrate-mediated proximity capture (SSPC) assay, for example.

Linear proximity generally refers to a linear base-pair distance, which may be assessed according to mapped distances in a reference genome, for example. Linear proximity distance may be provided as a distance between a 5′ or 3′ end of an ectopic portion and a 5′ or 3′ end of a gene and/or exon. An ectopic portion may be located at a position in linear proximity to the gene body of a gene, cancer gene, and/or oncogene when the ectopic portion is within about 1,000 base pairs, about 2,000 base pairs, about 3,000 base pairs, about 4,000 base pairs, about 5,000 base pairs, about 10.000 base pairs, about 20,000 base pairs, about 30,000 base pairs, about 40,000 base pairs, about 50,000 base pairs, about 60,000 base pairs, about 70,000 base pairs, about 80,000 base pairs, about 90,000 base pairs, about 100,000 base pairs, about 200,000 base pairs, about 300,000 base pairs, about 400,000 base pairs, about 500,000 base pairs, about 600,000 base pairs, about 700,000 base pairs, about 800,000 base pairs, about 900,000 base pairs, or about 1,000,000 base pairs of a gene body of a gene, cancer gene, and/or cancer gene. Sometimes the ectopic portion, while in proximity to a cancer gene or oncogene, as described above, also happens to be within a non-cancer gene/cancer gene. Sometimes the ectopic portion, while in proximity to a cancer gene or oncogene, as described above, is not within a gene and is positioned in an intergenic region.

In some embodiments, a structural variant comprises an ectopic portion of genomic DNA from a chromosome selected from the group consisting of: a chromosome listed in rows 16, 17, 22, and 23 of Table 8 (donor site). In some embodiments, an ectopic portion is located at a position in a chromosome selected from the group consisting of: a chromosome listed in rows 5, 6, 8, and 9 of Table 8 (receiver site) in proximity to a gene body for a corresponding cancer gene selected from the group consisting of: a cancer gene listed in row 7 of Table 8. In some embodiments, an ectopic portion is located at a position in a chromosome selected from the group consisting of: a chromosome listed in rows 5, 6, 8, and 9 of Table 8 (receiver site) in spatial proximity to a gene body for a corresponding cancer gene selected from the group consisting of: a cancer gene listed in row 7 of Table 8. In some embodiments, an ectopic portion is located at a position in a chromosome selected from the group consisting of: a chromosome listed in rows 5, 6, 8, and 9 of Table 8 (receiver site) in linear proximity to a gene body for a corresponding cancer gene selected from the group consisting of: a cancer gene fisted in row 7 of Table 8.

In some embodiments, an ectopic portion is located at a position in a chromosome selected from the group consisting of: a chromosome listed in rows 5, 6, 8, and 9 of Table 8 (receiver site) within about 1,000 base pairs, about 2,000 base pairs, about 3,000 base pairs, about 4,000 base pairs, about 5,000 base pairs, about 10.000 base pairs, about 20,000 base pairs, about 30,000 base pairs, about 40,000 base pairs, about 50,000 base pairs, about 60,000 base pairs, about 70,000 base pairs, about 80,000 base pairs, about 90,000 base pairs, about 100,000 base pairs, about 200,000 base pairs, about 300,000 base pairs, about 400,000 base pairs, about 500,000 base pairs, about 600,000 base pairs, about 700,000 base pairs, about 800,000 base pairs, about 900,000 base pairs, or about 1,000,000 base pairs of the gene body of the corresponding cancer gene selected from the group consisting of: a cancer gene listed in row 7 of Table 8. In some embodiments, an ectopic portion is located at a position in a chromosome selected from the group consisting of: a chromosome listed in rows 5, 6, 8, and 9 of Table 8 within a linear distance of the 5′ end of a corresponding cancer gene selected from the group consisting of: a cancer gene listed in row 7 of Table 8. The linear distance from the 5′ end for cancer gene is shown in row 12 of Table 8. In some embodiments the linear distance from the 5′ end can be about +/−10 bp, +1-50 bp, +/−100 bp, +/−500 bp, +/−1 kb, +/−5 kb, +/−10 kb, +/−50 kb, +/−100 kb or +/−500 kb what is listed in row 12 of Table 8.

In some embodiments, an ectopic portion is located at a position in a chromosome selected from the group consisting of: a chromosome listed in rows 5, 6, 8, and 9 of Table 8 within a linear distance of the 3′ end of a corresponding cancer gene selected from the group consisting of: a cancer gene listed in row 7 of Table 8. Row 13 of Table 8 shows the closest distance to the gene body of the corresponding cancer gene from row 7 of Table 8. If value in row 13 of Table 8 matches the value in row 12 of Table 8, the ectopic portion is nearer the 5′ of the corresponding cancer gene from row 7 of Table 8. If the value in row 13 of Table 8 does not match the value in row 12 of Table 8, the ectopic portion is nearer the 3′ of the corresponding cancer gene from row 7 of Table 8. If relevant (i.e. the values in row 12 and row 13 of Table 8 do not match), the linear distance from the 3′ end for cancer gene is shown in row 13 of Table 8. In some embodiments the linear distance from the 3′ end can be about −4-10 bp, +/−50 bp, +/−100 bp, +/−500 bp, +/−1 kb, +/−5 kb, +/−10 kb, +/−50 kb, +/−100 kb or +/−500 kb what is listed in row 13 of Table 8.

In some embodiments, a structural variant comprises an ectopic portion of genomic DNA from a chromosome selected from the group consisting of: a chromosome listed in rows 5, 6, 8, and 9 of Table 8 (donor site). In some embodiments, an ectopic portion is located at a position in a chromosome selected from the group consisting of: a chromosome listed in rows 16, 17, 22, and 23 of Table 8 (receiver site) in proximity to the gene body for a corresponding cancer gene selected from the group consisting of: a cancer gene listed in row 15 of Table 8. In some embodiments, an ectopic portion is located at a position in a chromosome selected from the group consisting of: a chromosome listed in rows 16, 17, 22, and 23 of Table 8 (receiver site) in spatial proximity to the gene body for a corresponding cancer gene selected from the group consisting of: a cancer gene listed in row 15 of Table 8. In some embodiments, an ectopic portion is located at a position in a chromosome selected from the group consisting of: a chromosome listed in rows 16, 17, 22, and 23 of Table 8 (receiver site) in linear proximity to the gene body for a corresponding cancer gene selected from the group consisting of: a cancer gene listed in row 15 of Table 8.

In some embodiments, an ectopic portion is located at a position in a chromosome selected from the group consisting of: a chromosome listed in rows 16, 17, 22, and 23 of Table 8 (receiver site) within about 1,000 base pairs, about 2,000 base pairs, about 3,000 base pairs, about 4,000 base pairs, about 5,000 base pairs, about 10,000 base pairs, about 20,000 base pairs, about 30,000 base pairs, about 40,000 base pairs, about 50,000 base pairs, about 60,000 base pairs, about 70,000 base pairs, about 80,000 base pairs, about 90,000 base pairs, about 100,000 base pairs, about 200,000 base pairs, about 300,000 base pairs, about 400,000 base pairs, about 500.000 base pairs, about 600,000 base pairs, about 700,000 base pairs, about 800,000 base pairs, about 900,000 base pairs, or about 1,000,000 base pairs of the gene body of the corresponding cancer gene selected from the group consisting of: a cancer gene listed in row 15 of Table 8. In some embodiments, an ectopic portion is located at a position in a chromosome selected from the group consisting of: a chromosome listed in rows 16, 17, 22, and 23 of Table 8 within a linear distance of the 5′ end of a corresponding cancer gene selected from the group consisting of: a cancer gene listed in row 15 of Table 8. The linear distance from the 5 end for cancer gene is shown in row 20 of Table 8. In some embodiments the linear distance from the 5′ end can be about +/−10 bp, +/−50 bp, +/−100 bp, +/−500 bp, +/−1 kb, +/−5 kb, +/−10 kb, +/−50 kb, +/−100 kb or +/−500 kb what is listed in row 20 of Table 8.

In some embodiments, an ectopic portion is located at a position in a chromosome selected from the group consisting of: a chromosome listed in rows 16, 17, 22, and 23 of Table 8 within a linear distance of the 3′ end of a corresponding cancer gene selected from the group consisting of: a cancer gene listed in row 15 of Table 8. Row 21 of Table 8 shows the closest distance to the gene body of the corresponding cancer gene from row 15 of Table 8. If value in row 21 of Table 8 matches the value in row 20 of Table 8, the ectopic portion is nearer the 5′ of the corresponding cancer gene from row 15 of Table 8. If the value in row 21 of Table 8 does not match the value in row 20 of Table 8, the ectopic portion is nearer the 3′ of the corresponding cancer gene from row 15 of Table 8. If relevant (i.e. the values in row 20 and row 21 of Table 8 do not match), the linear distance from the 3′ end for cancer gene is shown in row 21 of Table 8. In some embodiments the linear distance from the 3′ end can be about −4-10 bp, +/−50 bp, +/−100 bp, +/−500 bp, +/−1 kb, +/−5 kb, +/−10 kb, +/−50 kb, +/−100 kb or +/−500 kb what is listed in row 21 of Table 8. In certain aspects, the distance is a linear distance.

### Structural Variants

Provided herein are methods for treating a subject identified as having a structural variant associated with and/or adjacent to an HRR gene, such as can be found in Table 1. A structural variant may be referred to as a structural variation and/or a chromosomal rearrangement. A structural variant may comprise one or more of a translocation, inversion, insertion, deletion, and duplication. In some embodiments, a structural variant comprises a microduplication and/or a microdeletion. In some embodiments, a structural variant comprises a fusion (e.g., a gene fusion where a portion of a first gene is inserted into a portion of a second gene). Any type of structural variant, including, but not limited to, a translocation, insertion, inversion, deletion, duplication and the like, as described below, can be of any length and, in some embodiments, is about 1 base or base pair (bp) to about 250 megabases (Mb) in length. In some embodiments, a structural variation is about 1 base or base pair (bp) to about 50,000 kilobases (kb) in length (e.g., about 10 bp, 50 bp, 100 bp, 500 bp, 1 kb, 5 kb, 10 kb, 50 kb, 100 kb, 500 kb, 1000 kb, 5000 kb or 10,000 kb in length). A structural variant may be intra-chromosomal (rearrangement of genomic material within a chromosome) or inter-chromosomal (rearrangement of genomic material between two or more chromosomes).

A structural variant may comprise a translocation. A translocation is a genetic event that results in a rearrangement of chromosomal material. Translocations may include reciprocal translocations and Robertsonian translocations. A reciprocal translocation is a chromosome abnormality caused by exchange of parts between non-homologous chromosomes—two detached fragments of two different chromosomes are switched. A Robertsonian translocation occurs when two non-homologous chromosomes become attached, meaning that given two healthy pairs of chromosomes, one of each pair sticks and blends together homogeneously. A gene fusion may be created when a translocation joins two genes that are normally separate. Translocations may be balanced (i.e., in an even exchange of material with no genetic information extra or missing, sometimes with full functionality) or unbalanced (i.e., where the exchange of chromosome material is unequal resulting in extra or missing genes or fragments thereof).

A structural variant may comprise an inversion. An inversion is a chromosome rearrangement in which a segment of a chromosome is reversed end-to-end. An inversion may occur when a single chromosome undergoes breakage and rearrangement within itself. Inversions may be of two types: paracentric and pericentric. Paracentric inversions do not include the centromere, and both breaks occur in one arm of the chromosome. Pericentric inversions include the centromere, and there is a break point in each arm.

A structural variant may comprise an insertion. An insertion may be the addition of one or more nucleotide base pairs into a nucleic acid sequence. An insertion may be a microinsertion, e.g., generally a submicroscopic insertion of any length ranging from 1 base to about 10 megabases, such as from about 1 megabase to about 3 megabases. In certain embodiments, an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (e.g., insertion) of nucleic acid of unknown origin into a genome, chromosome, or segment thereof. In certain embodiments an insertion comprises the addition (e.g., insertion) of a single base.

A structural variant may comprise a deletion. In certain embodiments, a deletion is a genetic aberration in which a part of a chromosome or a sequence of DNA is missing. A deletion can, in certain embodiments, result in the loss of genetic material. In embodiments, a deletion can be translocated to another portion of the genome (balanced translocation or unbalanced translocation), such as on the same chromosome (same arm of the chromosome or other arm of the chromosome) or on a different chromosome. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can include a microdeletion, e.g., generally a submicroscopic deletion of any length ranging from 1 base to about 10 megabases, such as from about 1 megabase to about 3 megabases. A deletion can include the deletion of a single base.

A structural variant may comprise a duplication. In certain embodiments, a duplication is a genetic aberration in which a part of a chromosome or a sequence of DNA is copied and inserted back into the genome. In certain embodiments, a duplication is any duplication of a region of DNA. In some embodiments, a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can include a microduplication, e.g., generally a submicroscopic duplication of any length ranging from 1 base to about 10 megabases, such as from about 1 megabase to about 3 megabases. A duplication sometimes comprises one or more copies of a duplicated nucleic acid. A duplication may be characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications may occur as the result of an error in homologous recombination or due to a retrotransposon event.

A structural variant may be intra-chromosomal (rearrangement of genomic material within a chromosome) or inter-chromosomal (rearrangement of genomic material between two or more chromosomes). A structural variant may include a plurality of chromosomal rearrangements (e.g., translocations, inversions, insertions, deletions, duplications). For example, a structural variant may include a plurality of intra-chromosomal rearrangements. In certain instances, a structural variant may include a plurality of inter-chromosomal rearrangements. In certain instances, a structural variant may include a plurality of intra-chromosomal rearrangements and inter-chromosomal rearrangements.

A structural variant may include a plurality of chromosomal rearrangements (e.g., translocations, inversions, insertions, deletions, duplications). For example, a structural variant may include a plurality of intra-chromosomal rearrangements. In certain instances, a structural variant may include a plurality of inter-chromosomal rearrangements. In certain instances, a structural variant may include a plurality of intra-chromosomal rearrangements and inter-chromosomal rearrangements.

### Breakpoints and Donor/Receiver Sites

A structural variant may be defined according to one or more breakpoints. A breakpoint generally refers to a genomic position (i.e., genomic coordinate) where a structural variant occurs (e.g., translocation, inversion, insertion, deletion, or duplication). A breakpoint may refer to a genomic position where an ectopic portion of genomic material is inserted (e.g., a recipient site for an insertion or a translocation). A breakpoint may refer to a genomic position where a portion of genomic material is deleted (e.g., a donor site for an insertion or a translocation). A breakpoint may refer to a pair of genomic positions (i.e., genomic coordinates) that have become flanking (i.e., adjacent) to one another as a result of a structural variant (e.g., translocation, inversion, insertion, deletion, or duplication). A breakpoint may be defined in terms of a position or positions in a reference genome. A breakpoint may be defined in terms of a position or positions in a human reference genome (e.g., HG38 human reference genome). Generally, genomic positions discussed herein are in reference to an HG38 human reference genome, and corresponding and/or equivalent positions in any other human reference genome are contemplated herein.

A breakpoint may be defined in terms mapping to a position or positions in a reference genome. A breakpoint may be defined in terms of mapping to a position or positions in a human reference genome (e.g., HG38 human reference genome). A breakpoint may map to a position in a reference genome when a nucleic acid sequence located upstream, downstream, or spanning the breakpoint aligns with a corresponding sequence in a reference genome. Any suitable mapping method (e.g., process, algorithm, program, software, module, the like or combination thereof) can be used and certain aspects of mapping processes are described hereafter.

Mapping a nucleic acid sequence may comprise mapping one or more nucleic acid sequence reads (e.g., sequence information from a fragment whose physical genomic position is unknown), which can be performed in a number of ways, and often comprises alignment of the obtained sequence reads with a matching sequence in a reference genome. In such alignments, sequence reads generally are aligned to a reference sequence and those that align are designated as being “mapped”, “a mapped sequence read” or “a mapped read”.

The terms “aligned”, “alignment”, or “aligning” generally refer to two or more nucleic acid sequences that can be identified as a match (e.g., 100% identity) or partial match. Alignments can be done manually or by a computer (e.g., a software, program, module, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (e.g., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment contains a mismatch, such as a base not correctly paired with its canonical Watson-Crick base partner, e.g., A or T incorrectly paired with G or C. In some embodiments, an alignment comprises 1, 2, 3, 4 or 5 mismatches. Two or more sequences can be aligned using either strand. In certain embodiments a nucleic acid sequence is aligned with the reverse complement of another nucleic acid sequence. In certain instances, extra or missing bases within a sequence are expressed as gaps in an alignment and may or may not be factored into a percent identity calculation. For example, a percent identity calculation may include a number of mismatches and gaps or may include a number of mismatches only.

Various computational methods can be used to map and/or align sequence reads to a reference genome. Non-limiting examples of computer algorithms that can be used to align sequences include, without limitation, BLAST, BLITZ, FASTA, BOWTIE 1, BOWTIE 2, BWA, ELAND, MAQ, PROBEMATCH, SOAP or SEQMAP, or variations thereof or combinations thereof. In some embodiments, sequence reads can be aligned with reference sequences and/or sequences in a reference genome. In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database.

### Genes

A structural variant may be associated with one or more genes and/or genes associated with cancer, referred to herein in general as oncogenes. An oncogene can be any gene that, when altered, is associated with cancer. Alterations may include mutations, structural variants, copy number variations, and the like and combinations thereof. Alterations may be located within an oncogene (i.e., intragenic) or outside of/adjacent to an oncogene (i.e., intergenic, extragenic). In certain instances, alterations may be located within a different gene. Alterations may be located in a portion of genomic DNA that is proximal to a gene and/or an oncogene (e.g., within a certain linear proximity and/or within a certain spatial proximity). Alterations may affect expression of a gene and/or an oncogene (e.g., increased expression, decreased expression, no expression, constitutive expression). Alterations may affect the function of a protein encoded by the gene and/or oncogene (e.g., increased function, decreased function, loss-of-function, gain-of-function, constitutive function, change in function). Non-limiting examples of oncogenes are provided in Table 1 or 7.

In some embodiments, a structural variant is within a gene (e.g., within an intron and/or exon of a gene (e.g., an oncogene)). In some embodiments, a structural variant is outside of a gene (e.g., within an intergenic region or within a different nearby gene). In some embodiments, a structural variant is adjacent to a gene (e.g., within an intergenic region or within a different nearby gene). Thus, in some embodiments, a breakpoint for a structural variant is not within a gene (e.g., an oncogene). In certain instances, a structural variant (e.g., an intergenic structural variant) may be defined in terms of linear distance to a gene (e.g., an oncogene). Linear distance may be measured from the 5′ end of a gene and/or a 3 end of a gene. In some embodiments, a breakpoint for a structural variant may be located at least about 1 kb to about 500 kb from the 5′ end or 3′ end of a gene. For example, a breakpoint for a structural variant may be located at least about 1 kb, 2 kb, 3 kb, 4 kb, 5 kb, 10 kb, 20 kb, 30 kb, 40 kb, 50 kb, 60 kb, 70 kb, 80 kb, 90 kb, 100 kb, 200 kb, 300 kb, 400 kb, or 500 kb from the 5′ end or 3′ end of a gene.

Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), small interfering RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a nor-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil (U). Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single-stranded or double-stranded. Single-stranded DNA (ssDNA), for example, can be generated by denaturing double-stranded DNA by heating or by treatment with alkali, for example. Accordingly, in some embodiments, ssDNA is derived from double-stranded DNA (dsDNA).

Nucleic acid (e.g., genomic DNA, nucleic acid targets, oligonucleotides, probes, primers) may be described herein as being complementary to another nucleic acid, having a complementarity region, being capable of hybridizing to another nucleic acid, or having a hybridization region. The terms “complementary” or “complementarily” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions. In certain instances, a nucleotide sequence may be partially complementary to a target, in which not all nucleotides are complementary to every nucleotide in the target nucleic acid in all the corresponding positions.

The percent identity of two nucleotide sequences can be determined by aligning the sequences for optimal comparison purposes, e.g., when the total number of positions is different between the two nucleotide sequences, gaps may be introduced in the sequence of one or both sequences for optimal alignment. The nucleotides at corresponding positions are then compared, and the percent identity between the two sequences can be determined as a function of the number of identical positions shared by the sequences (e.g., % identity=#of identical positions/total #of positions×100). When a position in one sequence is occupied by the same nucleotide as the corresponding position in the other sequence, then the molecules are identical at that position. In certain instances, extra or missing bases within a sequence are expressed as gaps in an alignment and may or may not be factored into a percent identity calculation. For example, a percent identity calculation may include a number of mismatches and gaps or may include a number of mismatches only.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, oligonucleotide, or probe, to a nucleic acid molecule having a sequence complementary to the primer, oligonucleotide, or probe compared to hybridization to a nucleic acid molecule not having a complementary sequence. For example, specific hybridization includes the hybridization of a primer, oligonucleotide, or probe to a target nucleic acid sequence that is complementary to the primer, oligonucleotide, or probe.

Primer, oligonucleotide, or probe sequences and length can affect hybridization to target nucleic acid sequences. Depending on the degree of mismatch between the primer, oligonucleotide, or probe and target nucleic acid, low, medium or high stringency conditions may be used to effect primer/target, oligonucleotide/target, or probe/target annealing. As used herein, the term “stringent conditions” refers to conditions for hybridization and washing. Methods for hybridization reaction temperature condition optimization are known, and can be found. e.g., in Current Protocols in Molecular Biology, John Wiley & Sons, N.Y., 6.3.1-6.3.6 (1989), as described further below.

In some embodiments, target nucleic acids comprise degraded DNA. Degraded DNA may be referred to as low-quality DNA or highly degraded DNA. Degraded DNA may be highly fragmented, and may include damage such as base analogs and abasic sites subject to miscoding lesions and/or intermolecular crosslinking. For example, sequencing errors resulting from deamination of cytosine residues may be present in certain sequences obtained from degraded DNA (e.g., miscoding of C to T and G to A).

Nucleic acid may be derived from one or more sources (e.g., a biological sample described herein) by methods known in the art. Any suitable method can be used for isolating, extracting and/or purifying DNA from a biological sample (e.g., from blood or a blood product, tissue, tumor), non-limiting examples of which include methods of DNA preparation, various commercially available reagents or kits, such as DNeasy®, RNeasy®, QIAprep®, QIAquick®, and QIAamp® (e.g., QIAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md); GenomicPrep™ Blood DNA Isolation Kit (Promega, Madison, Wis.); GFX™ Genomic Blood DNA Purification Kit (Amersham, Piscataway, N.J.); DNAzol®, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA); the like or combinations thereof. In certain aspects, nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccfDNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type II cell death, including mitotic, oncotic, toxic, ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In certain aspects, cell-free nucleic acid comprises circulating cancer nucleic acid (e.g., cancer DNA). In certain aspects, cell-free nucleic acid comprises circulating tumor nucleic acid (e.g., tumor DNA).

Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In some instances, cancer nucleic acid and/or tumor nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, or tumor nucleic acid).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

### Nucleic Acid Analysis

A method herein may comprise one or more nucleic acid analyses. For example, nucleic acid obtained from a sample from a subject may be analyzed for the presence or absence of a structural variant. Any suitable process for detecting a structural variant in a nucleic acid sample may be used. Non-limiting examples of processes for analyzing nucleic acid include amplification (e.g., polymerase chain reaction (PCR)), targeted sequencing, microarray, and fluorescence in situ hybridization (FISH), methods that preserve spatial-proximal relationships and/or spatial-proximal contiguity information, and methods that generate proximity ligated nucleic acid molecules.

In some embodiments, a nucleic acid analysis comprises nucleic acid amplification. For example, nucleic acids may be amplified under amplification conditions. The term “amplified” or “amplification” or “amplification conditions” generally refer to subjecting a target nucleic acid in a sample to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR). Detecting a structural variant (SV) described herein using amplification (e.g., PCR) may include use of primers designed to hybridize to a region upstream (e.g., 5′) of one or more SV breakpoints, hybridize to a region downstream (e.g., 3′) of one or more SV breakpoints, hybridize to a region associated with and/or adjacent to one or more SV breakpoints, and/or hybridize to a region spanning one or more SV breakpoints. Examples of PCR primers useful for identifying a structural variant are provided herein.

In some embodiments, a nucleic acid analysis comprises fluorescence in situ hybridization (FISH). Fluorescence in situ hybridization (FISH) is a technique that uses fluorescent probes that bind to a nucleic acid sequence with a high degree of sequence complementarity. In certain configurations, fluorescence microscopy may be used to observe where the fluorescent probe is bound to a chromosome. Detecting a structural variant (SV) described herein using fluorescence in situ hybridization (FISH) may include use of probes designed to hybridize to a region upstream (e.g., 5′) of one or more SV breakpoints, hybridize to a region downstream (e.g., 3′) of one or more SV breakpoints, hybridize to a region associated with and/or adjacent to one or more SV breakpoints, and/or hybridize to a region spanning one or more SV breakpoints. Examples of probes useful for identifying a structural variant are provided herein.

In some embodiments, a nucleic acid analysis comprises a microarray (e.g., a DNA microarray, DNA chip, biochip). A DNA microarray is a collection of DNA probes attached to a solid surface. Probes can be short sections of a gene or other genomic DNA element that can hybridize to target nucleic acids in a sample (e.g., under high-stringency conditions). Probe-target hybridization is usually detected and quantified by detection of fluorophore-, silver-, or chemiluminescence-labeled targets to determine presence, absence, and/or relative abundance of target nucleic acid sequences in the sample. Detecting a structural variant (SV) described herein using DNA microarrays may include use of array probes designed to hybridize to a region upstream (e.g., 5′) of one or more SV breakpoints, hybridize to a region downstream (e.g., 3′) of one or more SV breakpoints, hybridize to a region associated with and/or adjacent to one or more SV breakpoints, and/or hybridize to a region spanning one or more SV breakpoints. Examples of array probes useful for identifying a structural variant are provided herein.

In some embodiments, a nucleic acid analysis comprises sequencing (e.g., genome-wide sequencing, targeted sequencing). Nucleic acid can be sequenced using any suitable sequencing platform, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In embodiments, a high-throughput sequencing method can be used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In embodiments, MPS sequencing methods utilize a targeted approach, where specific chromosomes, genes or regions of interest are sequenced.

Non-limiting examples of sequencing platforms include a sequencing platform provided by Illumina® (e.g., HiSeq™, HiSeq™ 2000, MiSeq™, Genome Analyzer™, and Genome Analyzer™ II sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLiD sequencing system); Roche (e.g., 454 GS FLX+ and/or GS Junior sequencing systems); Helicos True Single Molecule Sequencing; Ion semiconductor-based sequencing (e.g., as developed by Life Technologies), WildFire, 5500, 5500xl W and/or 5500xl W Genetic Analyzer based technologies (e.g., as developed and sold by Life Technologies. U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing; or any other suitable sequencing platform. Other sequencing methods that can be used to conduct methods herein include digital PCR, sequencing by hybridization, nanopore sequencing, chromosome-specific sequencing (e.g., using DANSR (digital analysis of selected regions) technology).

In certain embodiments, the sequencing process is a highly multiplexed sequencing process. In certain instances, a full or substantially full sequence is obtained and sometimes a partial sequence is obtained.

For targeted sequencing, a target nucleic acid may be amplified (e.g., by PCR with primers specific to the target), enriched using a probe-based approach, where one or more probes hybridize to a target nucleic acid prior to sequencing, or enriched using Cas9-mediated approaches, such as Cas9-guided adapter ligation, as described in Gilpatrick. T. et al., Targeted nanopore sequencing with Cas9-guided adapter ligation, Nature Biotechnology, volume 38, pages 433-438 (2020). Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides. The length of a sequence read often is associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments, sequence reads are of a mean, median, average or absolute length of about 100 bp to about 200 bp.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid.

In some embodiments, a nucleic acid analysis comprises a method that preserves spatial-proximal relationships and/or spatial proximal contiguity information (see e.g., International PCT Application Publication No. WO2019/104034; International PCT Application Publication No. WO2020/106776; International PCT Application Publication No. WO2020236851; Kempfer, R., & Pombo, A. (2019). Methods for mapping 3D chromosome architecture. Nature Reviews Genetics. doi:10.1038/s41576-019-0195-2; and Schmitt, Anthony D.; Hu, Ming; Ren, Bing (2016). Genome-wide mapping and analysis of chromosome architecture. Nature Reviews Molecular Cell Biology. doi:10.1038/nrm.2016.104; each of which is incorporated by reference in its entirety, to the extent permitted by law). Methods that preserve spatial-proximal relationships and/or spatial proximal contiguity information generally refer to methods that capture and preserve the native spatial conformation exhibited by nucleic acids when associated with proteins as in chromatin and/or as part of a nuclear matrix. Spatial-proximal contiguity information can be preserved by proximity ligation, by solid substrate-mediated proximity capture (SSPC), by compartmentalization with or without a solid substrate or by use of a Tn5 tetramer. Methods that preserve spatial-proximal contiguity information may be based on proximity ligation or may be based on a different principle where special proximity is inferred. Methods based on proximity ligation may include, for example, 3C, 4C, 5C, Hi-C, TCC, GCC, TLA, PLAC-seq, HiChIP, ChIA-PET, Capture-C, Capture-HiC, single-cell HiC, sciHiC, single-cell 3C, single-cell methyl-3C, DNAase HiC, Micro-C, Tiled-C, and Low-C. Methods where special proximity is inferred based on a principle other than proximity ligation may include, for example, SPRITE, scSPRITE, Genome Architecture Mapping (GAM), ChIA-Drop, imaging-based approaches using labeled probes and visualization of DNA, and plus/minus sequencing of an imaged sample (e.g. in situ Genome Sequencing (IGS)). In some embodiments, a nucleic acid analysis comprises generating proximity ligated nucleic acid molecules (e.g., using a method described herein). In some embodiments, a nucleic acid analysis comprises sequencing the proximity ligated nucleic acid molecules, e.g., by a suitable sequencing process known in the art or described herein.

### Non-Spatial Proximal Contiguity DNA Sequencing Methodologies:

Non-spatial proximal contiguity sequencing methodologies, including but not limited to Shotgun WGS, Linked-Read WGS and other forms of synthetic long-read sequencing, Mate-pair WGS and similar techniques (Fosmids, BACs), Long-read WGS, and other known or anticipated non-spatial proximal contiguity DNA sequencing methodologies, either sequenced “in bulk” or with single-cell and/or spatial resolution, either in “genome-wide” or “targeted” format (“targeted” meaning, for example, by using known or anticipated target enrichment methodologies (e.g. probe based enrichment or PCR), or depletion methodologies (e.g. using CRISPR), or other targeted sequencing techniques (e.g. adaptive sampling), and either sequenced on any known or anticipated short or long-read sequencing platform.

### Spatial Proximal Contiguity DNA Sequencing Methodologies:

### Proximity Ligation DNA Sequencing:

Genome-wide proximity ligation sequencing techniques, including but not limited to: 3C-seq, Hi-C, DNAase HiC, Micro-C, Low-C, TCC, GCC, single-cell HiC, sciHiC, single-cell 3C, single-cell methyl-3C and other genome-wide bulk or single-cell and/or spatial derivatives, sequenced on any known or anticipated short or long-read sequencing platforms.

Targeted proximity ligation sequencing techniques, including but not limited to 3C-(q)PCR, 4C, 5C, Targeted Locus Amplification, PLAC-seq, HiChIP, ChIA-PET, Capture-C, Capture-HiC, Tiled-C and other genome-wide bulk or single-cell or spatial derivatives, including additional “targeted” techniques (“targeted” meaning, for example, by using known or anticipated target enrichment methodologies (e.g. probe based enrichment or PCR, or protein enrichment), or depletion methodologies (e.g. using CRISPR), or other targeted sequencing techniques (e.g. adaptive sampling), and sequenced on any known or anticipated short or long-read sequencing platforms.

### Non-Proximity Ligation DNA Sequencing:

Non-proximity ligation sequencing techniques, including but not limited to: SPRITE, scSPRITE, other SPRITE derivatives or related techniques involving barcoding of chromatin aggregates, ChIA-Drop or other droplet-based chromatin aggregate barcoding and sequencing techniques, and Genome Architecture Mapping or related techniques where spatial proximal contiguity is inferred from co-occurrence in cryosections. In addition, it is anticipated that additional derivatives of the above may be suitable for proximity fusion detection (i.e. finding fusions adjacent to a cancer gene), including “targeted” versions (“targeted” meaning, for example, by using known or anticipated target enrichment methodologies (e.g. probe based enrichment or PCR), or depletion methodologies (e.g. using CRISPR), or other targeted sequencing techniques (e.g. adaptive sampling), and sequenced on any known or anticipated short or long-read sequencing platforms.

### Imaging Methodologies:

Classic DNA FISH analysis, with one probe on either side of a breakpoint, can detect proximity fusions. However, recent derivatives thereof, including but not limited to SeqFISH, MERFISH, and OligoFISSEQ, could also detect proximity fusions, and due to their high plexity capability could be more tolerant to heterogeneous breakpoint locations and be able to detect proximity fusions involving more than one gene per experiment (possibly hundreds of genes or someday genome-scale).

### Imaging Plus Sequencing Methodologies:

In situ Genome Sequencing (IGS), or related techniques that sequence DNA molecules “in situ”, measuring the location in the nucleus of each sequenced DNA molecule.

### Optical Genome Mapping

PCR—As an example, breakpoint-crossing PCR could be used to detect proximity fusions, so long as the breakpoint is flanked by PCR primers.

Methodologies that infer breakpoints based on genomic coverage—in the absence of identifying a sequence fragment that contains a genomic breakpoint of a proximity (or gene) fusion, techniques may be used to infer structural variant breakpoints based on genomic coverage alone. For example, cytogenic microarrays (e.g. including but not limited to array-based CGH, SNP microarrays, or DNA methylation arrays) can be used to identify copy number gains and losses (i.e. unbalanced chromosomal rearrangements), and the genomic positions where the copy number gain or loss starts/ends can be inferred to be a structural variant breakpoint. One then may be able to look for cancer genes near those breakpoints to identify proximity fusions. While the description here uses microarrays as an example methodology for generating genomic coverage data, it is anticipated that essentially any of the above described sequencing-based methodologies (Non-spatial proximal contiguity DNA Sequencing Methodologies, Spatial proximal contiguity DNA Sequencing Methodologies, Imaging plus Sequencing Methodologies), or Optical Genome Mapping, or any technique that reliably quantifies genome coverage could potentially be used to infer breakpoints based on coverage, and potentially enable the detection of proximity fusions in the absence of a analyzed DNA fragment containing a breakpoint.

In some embodiments, a nucleic acid analysis comprises a method for preparing nucleic acids from particular types of samples that preserves spatial-proximal relationships and/or spatial-proximal contiguity information in the sequence of the nucleic acids. Nucleic acid molecules that preserve spatial-proximal contiguity information can fragmented and sequenced using short-read sequencing methods (e.g., Illumina, nucleic acid fragments of lengths approximately 500 bp) or intact molecules that preserve spatial-proximal contiguity information can be sequenced using long-read sequencing (e.g., Illumina, Oxford Nanopore, or others, nucleic acid fragments of lengths approximately 30 K bp or greater).

In certain embodiments, a sample can be a fixed sample that is embedded in a material such as paraffin (wax). In some embodiments, a sample can be a formalin fixed sample. In certain embodiments, a sample is formalin-fixed paraffin-embedded (FFPE) sample. In some embodiments, a formalin-fixed paraffin-embedded sample can be a tissue sample or a cell culture sample. In some embodiments, a tissue sample has been excised from a patient and can be diseased or damaged. In some embodiments, a tissue sample is not known to be diseased or damaged. In certain embodiments, a formalin-fixed paraffin-embedded sample can be a formalin-fixed paraffin-embedded section, block, scroll or slide. In certain embodiments, a sample can be a deeply formalin-fixed sample, as described below.

In certain embodiments, a formalin-fixed paraffin-embedded sample is provided on a solid surface and a method of preparing nucleic acid that preserves spatial-proximal contiguity information is performed on the solid surface. In some embodiments, a solid surface is a pathology slide. In some embodiments, additional downstream reactions are also performed on the solid surface.

Those of skill in the art are familiar with methods that can be substituted for steps requiring centrifugation and that achieve a comparable result but are performed on a solid surface.

In some embodiments, methods that preserve spatial-proximal contiguity information comprise methods that generate proximity ligated nucleic acid molecules (e.g., using proximity ligation). A proximity ligation method is one in which natively occurring spatially proximal nucleic acid molecules are captured by ligation to generate ligated products. Proximity ligation methods generally capture spatial-proximal contiguity information in the form of ligation products, whereby a ligation junction is formed between two natively spatially proximal nucleic acids. Once the ligation products are formed, the spatial-proximal contiguity information is detected using next generation sequencing, whereby one or more ligation junctions (either from an entire ligation product or fragment of a ligation product) are sequenced (as described herein). With this sequence information, one is informed that the nucleic acid molecules from a given ligation product (or ligation junction) are natively spatially proximal nucleic acids. In some embodiments, reagents that generate proximity ligated nucleic acid molecules can include a restriction endonuclease, a DNA polymerase, a plurality of nucleotides comprising at least one biotinylated nucleotide, and a ligase. In certain embodiments, two or more restriction endonucleases are used.

Any suitable method for carrying out proximity ligation may be used. For example, a HiC method typically includes the following steps: (1) digestion of chromatin of a solubilized and decompacted FFPE sample with a restriction enzyme (or fragmentation); (2) labelling the digested ends by filling in the 5′-overhangs with biotinylated nucleotides; and (3) ligating the spatially proximal digested ends, thus preserving spatial-proximal contiguity information. Once spatial-proximal contiguity information is preserved, further steps in a HiC method may include: purifying and enriching biotin-labelled ligation junction fragments, preparing a library from the enriched fragments and sequencing the library. Another example of a proximity ligation method may include the following steps: (1) digestion of chromatin of the solubilized and decompacted sample with a restriction enzyme (or fragmentation); (2) blunting the digested or fragmented ends or omission of the blunting procedure; and (3) ligating the spatially proximal ends, thus preserving spatial-proximal contiguity information. Once spatial-proximal contiguity information is preserved, further steps can include: using size selection to purify and enrich ligated fragments, which represent ligation junction fragments, preparing a library from the enriched fragments and sequencing the library. In some embodiments, proximity ligated nucleic acid molecules are generated in situ (i.e., within a nucleus). For methods that include Capture HiC, a further step is included where ligation products containing certain nucleic acid sequences are enriched using one or more capture probes (see, e.g., International Patent Application Publication No. WO 2014/168575). A capture probe generally includes a short sequence of nucleotides or oligonucleotide (e.g., 10-500 bases in length) capable of hybridizing to another nucleotide sequence. In some embodiments, a capture probe includes a label, e.g., a label for selectively purifying specific nucleic acid sequences of interest. Labels are discussed herein and can include, for example, a biotin or digoxigenin label. In some embodiments, capture probes are designed according to a panel of sequences and/or genes of interest (e.g., an oncopanel provided herein).

### Oligonucleotides

Provided herein are oligonucleotides for analyzing structural variants associated with and/or adjacent to a HRR gene, whereby subjects having one or more structural variants are selected for treatment. Oligonucleotides may be artificially synthesized. Accordingly, provided herein in certain embodiments are synthetic oligonucleotides. An oligonucleotide generally refers to a nucleic acid (e.g., DNA, RNA) polymer that is distinct from a target nucleic acid (e.g., a target nucleic acid comprising one or more structural variants described herein), and may be referred to as oligos, probes, and/or primers. Oligonucleotides may be short in length (e.g., less than 50 bp, less than 40 bp, less than 30 bp, less than 20 bp, less than 10 bp). In some embodiments, oligonucleotides are between about 10 to about 500 consecutive nucleotides in length. For example, an oligonucleotide may be about 10, 20, 30, 40, 50, 60, 70, 80, 90, 100, 150, 200, 250, 300, 350, 400, 450, or 500 consecutive nucleotides in length.

Oligonucleotides may be designed to hybridize to a region of a sample nucleic acid that is proximal to, adjacent to, and/or spanning a structural variant described herein, or portion thereof. Oligonucleotides may be designed to hybridize to a region of a sample nucleic acid that comprises a receiving site, a donor site, or a combination of a receiving site and a donor site.

Oligonucleotides may include probes and/or primers useful for detecting presence, absence, or amount of a structural variant in a nucleic acid sample. Probes and/or primers may be used in conjunction with any suitable nucleic acid analysis (e.g., a nucleic acid analysis method described herein). For example, probes and/or primers may be used in an amplification process (e.g., PCR, quantitative PCR), FISH (e.g., labeled FISH probes, labeled FISH probe pairs (e.g., with fluorophore and quencher)), microarray, nucleic acid capture, nucleic acid enrichment, nucleic acid sequencing, and the like. Oligonucleotides may be designed to hybridize to a portion or portions of a genome that is/are proximal to, adjacent to, overlapping, partially overlapping, or spanning a structural variant or portion thereof.

Oligonucleotides may include a probe or primer capable of hybridizing to a region of a first breakpoint and a region of a second breakpoint of a structural variant described herein.

Accordingly, such probes and primers comprise a first sequence complementary to a receiving site in a structural variant and a second sequence complementary to a donor site in a structural variant. Such probes and primers are useful for detecting the presence, absence, or amount of a structural variant in a sample, for example, by way of hybridizing to the sample nucleic acid when the structural variant is present and not hybridizing to the sample nucleic acid when the structural variant is absent.

In some embodiments, an oligonucleotide comprises (i) a first polynucleotide identical to or complementary to a subsequence of 5 or more consecutive nucleotides in length within a region of a chromosome comprising a receiving site for a structural variant described herein, and (ii) a second polynucleotide identical to or complementary to a subsequence of about 5 or more consecutive nucleotides in length within a region of a chromosome comprising a donor site for a structural variant described herein. Such oligonucleotide can specifically hybridize (e.g., under stringent hybridization conditions) to a target sequence comprising the subsequence of (i) and the subsequence of (ii). Methods for hybridization reaction temperature condition optimization are known, and can be found, e.g., in Current Protocols in Molecular Biology, John Wiley & Sons, N.Y., 6.3.1-6.3.6 (1989). Aqueous and non-aqueous methods are described in the aforementioned reference and either can be used. Non-limiting examples of stringent hybridization conditions include, for example, hybridization in 6× sodium chloride/sodium citrate (SSC) at about 45° C., followed by one or more washes in 0.2×SSC, 0.1% SDS at 50° C. Another example of stringent hybridization conditions includes hybridization in 6× sodium chloride/sodium citrate (SSC) at about 45° C., followed by one or more washes in 0.2×SSC, 0.1% SDS at 55° C. A further example of stringent hybridization conditions includes hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions can include 0.5 M sodium phosphate, 7% SDS at 65° C., followed by one or more washes at 0.2×SSC, 1% SDS at 65° C. Stringent hybridization temperatures also can be altered (generally, lowered) with the addition of certain organic solvents, such as formamide for example. Organic solvents such as formamide can reduce the thermal stability of double-stranded polynucleotides, so that hybridization can be performed at lower temperatures, while still maintaining stringent conditions and extending the useful life of heat labile nucleic acids

In some embodiments, an oligonucleotide comprises (i) a first polynucleotide identical to or complementary to a subsequence of 5 or more consecutive nucleotides in length within a region of a chromosome, where the region spans positions selected from the group of row 5 and row 6 of Table 8; and (ii) a second polynucleotide identical to or complementary to a subsequence of about 5 or more consecutive nucleotides in length within a region of a chromosome, where the region spans positions selected from the group of row 22 and row 23 of Table 8. The oligonucleotide may specifically hybridize (e.g., under stringent hybridization conditions) to a target sequence comprising the subsequence of (i) and the subsequence of (ii).

Oligonucleotides may include a pair of probes or primers capable of hybridizing to a region of a first breakpoint and a region of a second breakpoint of a structural variant described herein. Accordingly, such probe and primer pairs comprise a first member complementary to a receiving site in a structural variant and a second member complementary to a donor site in a structural variant. Such probes and primers may be useful for detecting the presence or absence of a structural variant in a sample, for example, by way of hybridizing to the sample nucleic acid at specific locations when the structural variant is present and hybridizing to the sample nucleic acid at different locations when the structural variant is absent.

In some embodiments, a composition comprises (a) a first oligonucleotide comprising a first polynucleotide identical to or complementary to a subsequence of 5 or more consecutive nucleotides in length within a region of a chromosome, where the region spans positions selected from the group of row 5 and row 6 of Table 8; and (b) a second oligonucleotide comprising a second polynucleotide identical to or complementary to a subsequence of about 5 or more consecutive nucleotides in within a region of a chromosome, where the region spans positions selected from the group of row 22 and row 23 of Table 8, a composition comprises (a) a first oligonucleotide comprising a first polynucleotide identical to or complementary to a subsequence of 5 or more consecutive nucleotides in length within a region of a chromosome, where the region spans positions selected from the group of row 5 and row 6 of Table 8; and (b) a second oligonucleotide comprising a second polynucleotide identical to or complementary to a subsequence of about 5 or more consecutive nucleotides in length within a region of a chromosome, where the region spans positions selected from the group of row 22 and row 23 of Table 8. The first oligonucleotide may specifically hybridize (e.g., under stringent hybridization conditions) to a target nucleic acid comprising the subsequence of (a). The second oligonucleotide may specifically hybridize (e.g., under stringent hybridization conditions) to a target nucleic acid comprising the subsequence of chromosome 9 in (b). In some embodiments, the first oligonucleotide specifically hybridizes (e.g., under stringent hybridization conditions) to a target nucleic acid comprising the subsequence of chromosome 9 in (a) and does not specifically hybridize to a target nucleic acid comprising the subsequence in (b). In some embodiments, the second oligonucleotide specifically hybridizes (e.g., under stringent hybridization conditions) to a target nucleic acid comprising the subsequence of (b) and does not specifically hybridize to a target nucleic acid comprising the subsequence of (a).

### Samples

Provided herein are methods and compositions for processing and/or analyzing nucleic acid. Nucleic acid utilized in methods and compositions described herein may be isolated from a sample obtained from a subject (e.g., a test subject). A subject can be any living or non-living organism, including but not limited to a human and a non-human animal. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a human. A subject may be a male or female. A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. In some embodiments, a subject is an adult patient. In some embodiments, a subject is a pediatric patient.

A nucleic acid sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism. In some embodiments, a nucleic acid sample is isolated or obtained from a cell(s), tissue, organ, and/or the like of an animal (e.g., an animal subject). In some instances, a nucleic acid sample may be obtained as part of a diagnostic analysis.

A sample or test sample may be any specimen that is isolated or obtained from a subject or part thereof (e.g., a human subject, a cancer patient, a tumor). Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, cancer cells may be included in the sample.

A sample can be a liquid sample. A liquid sample can comprise extracellular nucleic acid (e.g., circulating cell-free DNA). Examples of liquid samples include, but are not limited to, blood or a blood product (e.g., serum, plasma, or the like), urine, cerebrospinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells. B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of tumor or cancer DNA found in a patient's blood, for example, may be performed using, e.g., whole blood, serum, or plasma. Methods for preparing serum or plasma from blood obtained from a subject (e.g., patient; cancer patient) are known.

For example, a subject's blood (e.g., patient's blood; cancer patient's blood) can be placed in a tube containing EDTA or a specialized commercial product such as Cell-Free DNA BCT (Streck, Omaha, NE) or Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

A sample may be a tumor nucleic acid sample (i.e., a nucleic acid sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation.

In some embodiments, a sample is a tissue sample, a cell sample, a blood sample, or a urine sample. In some embodiments, a sample comprises formalin-fixed, paraffin-embedded (FFPE) tissue. In some embodiments, a sample comprises frozen tissue. In some embodiments, a sample comprises peripheral blood. In some embodiments, a sample comprises blood obtained from bone marrow. In some embodiments, a sample comprises cells obtained from urine. In some embodiments, a sample comprises cell-free nucleic acid. In some embodiments, a sample comprises one or more tumor cells. In some embodiments, a sample comprises one or more circulating tumor cells. In some embodiments, a sample comprises a solid tumor. In some embodiments, a sample comprises a blood tumor.

### Cancers

In some embodiments, a subject has, or is suspected of having, a disease. In some embodiments, a subject has, or is suspected of having, cancer. In some embodiments, a subject has, or is suspected of having, a cancer associated with one or more genes and/or oncogenes described herein. For example, in some embodiments, a subject has, or is suspected of having, a cancer associated with one or more genes and/or oncogenes chosen from the HRR genes in Table 1. In some embodiments, a subject has, or is suspected of having, a cancer associated with one or more structural variants described herein.

Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, a chordoma, a salivary gland carcinoma, kidney cancer, liver cancer, prostate cancer, vulval cancer, thyroid cancer, hepatic carcinoma, various types of head and neck cancer, and the like. In some embodiments, a cancer is a rare cancer. In some embodiments, a cancer is kidney cancer, breast cancer, colorectal cancer, gastric cancer, lung cancer, thyroid cancer, or testicular cancer. In certain embodiments, the cancer is any one of the types listed in Row 3 of Table 8. In some embodiments, a cancer is glioma. In some embodiments, a cancer is glioblastoma. In some embodiments, a cancer is a glioblastoma. In embodiments, a cancer is pediatric glioblastoma. In certain embodiments, the glioblastoma is a newly diagnosed. In embodiments, the glioblastoma is recurrent.

### Diagnosis and Treatment

In some embodiments, a method herein comprises providing a diagnosis and/or a likelihood of cancer in a subject. A diagnosis and/or likelihood of cancer may be provided when the presence of a structural variant described herein is detected. In some embodiments, a method herein comprises performing a further test (e.g., biopsy, blood test, imaging) to confirm a cancer diagnosis.

In some embodiments, a method herein comprises administering a treatment to a subject. A treatment may be administered to a subject when the presence of a structural variant described herein is detected. Suitable treatments may be determined by a physician and may include one or more modulators (e.g., activators, blockers) of one or more genes, proteins, oncogenes, oncoproteins (proteins encoded by oncogenes), and/or oncogene-related components associated with a detected structural variant.

An oncogene-related component generally refers to one or more components chosen from among (i) an oncogene, including exons, introns, and 5′ (upstream), e.g. promoter regions, or 3′ (downstream) regulatory elements; (ii) transcription products, mRNA, or cDNA: (iii) translation products, protein, gene products, or gene expression products, or homologs of, synthetic versions of, analogs of, receptors of, agonists to receptors of, antagonists to receptors of, upstream pathway regulators of, or downstream pathway targets of translation products, protein, gene products, or gene expression products; and (iv) any component that could be considered by one skilled in the art as a target for a modulator (e.g., activator, blocker, drug, medicament).

The term “modulator” of a gene (or oncogene) or “gene/oncogene modulator” includes modulation of the gene/oncogene, modulation of a protein encoded by the gene/oncogene and/or modulation of a gene/oncogene-related component, such as a component of the pathway through which the gene/oncogene mediates its effects, a promoter, an enhancer, and the like.

A modulator generally refers to an agent that is capable of changing an activity (e.g., change in level and/or nature of an activity) of a component in a system, compared to a component's level and/or activity under otherwise comparable conditions when the modulator is absent. A modulator herein may refer to an agent that is capable of changing an activity (e.g., change in level and/or nature of an activity) of a gene, protein, an oncogene, oncoprotein, and/or oncogene-related component in a system compared to a gene's, a protein's, an oncogene's, oncoprotein's, and/or oncogene-related component's level and/or activity under otherwise comparable conditions when the modulator is absent. In some embodiments, a modulator is an activator, in that level and/or activity is increased in its presence as compared with that observed under otherwise comparable conditions when the modulator is absent. In some embodiments, a modulator is an inhibitor, in that level and/or activity is reduced in its presence as compared with otherwise comparable conditions when the modulator is absent. In some embodiments, a modulator interacts directly with a target component of interest. In some embodiments, a modulator interacts indirectly (e.g., directly with an intermediate agent that interacts with the target component) with a target component of interest. In some embodiments, a modulator affects the level of a target component of interest, as one non-limiting example, by impacting an upstream signaling pathway associated with the target component of interest. In some embodiments, a modulator affects an activity of a target component of interest without affecting a level of the target component, as one non-limiting example, by impacting a downstream signaling pathway associated with the target component of interest. In some embodiments, a modulator affects both level and activity of a target component of interest, such that an observed difference in activity is not entirely explained by or commensurate with an observed difference in level.

The term “modulator of [oncogene]” or “[oncogene] modulator” means “modulator of [oncogene], modulator of [onco]protein, and/or [oncogene]-related components” or “[oncogene], [onco]protein, and/or [oncogene]-related components modulator,” respectively, where [oncogene] can mean any oncogene identified herein. In certain embodiments, the oncogene is selected from the HRR genes shown in Table 1. In other embodiments, the oncogene is selected from the HRR genes shown in Row 3 of Table 8.

In certain embodiments, the subject having cancer and identified as having a structural variant associated with and/or adjacent to an HRR gene is treated with a standard of care treatment for the cancer and, additionally, is treated with a homologous recombination deficient direct therapy, such as an inhibitor of a polyadenosine diphosphate-ribose polymerase (PARP) enzyme. In embodiments, the subject having cancer and identified as having a structural variant associated with an HRR gene is treated with a homologous recombination deficient direct therapy, such as an inhibitor of a polyadenosine diphosphate-ribose polymerase (PARP) enzyme, instead of treatment with a standard of care treatment. The subject identified as having a structural variant associated with and/or adjacent to an HRR gene, and treated with a homologous recombination deficient direct therapy, such as an inhibitor of a polyadenosine diphosphate-ribose polymerase (PARP) enzyme, can be a subject that previously was undergoing a standard of care treatment or can be a subject not previously undergoing a standard of care treatment (i.e., the first line of treatment administered is a homologous recombination deficient direct therapy, such as an inhibitor of a polyadenosine diphosphate-ribose polymerase (PARP) enzyme). Treatment regimens also can be modified, e.g., to incorporate portions of a standard of care treatment regimen along with a homologous recombination deficient direct therapy, such as an inhibitor of a polyadenosine diphosphate-ribose polymerase (PARP) enzyme.

A “standard of care treatment,” or “standard treatment,” as used herein, is a treatment that generally is accepted by medical experts and/or health professionals as being the most appropriate for a certain type of disease in a certain setting and/or based on the age, gender, coexisting health conditions, stage of the disease, e.g., cancer, and the like. A standard of care treatment can, in certain aspects, be FDA approved or at a Phase II or greater stage of FDA approval (e.g., for experimental therapies in subjects selected for clinical trials).

Standard of care treatments for cancers are known and described, for example by guidelines issued by NATIONAL COMPREHENSIVE CANCER NETWORK™. Current guidelines can be accessed at: https://www.nccn.org/guidelines/category_1. In embodiments, the subject having cancer and identified as having a structural variant associated with and/or adjacent to or within an HRR gene is refractory to a standard of care treatment for the cancer, e.g., is refractory to a standard of care treatment for glioma. The term “refractory,” as used herein, means that progression of the disease is observed during or following one or more cycles, or a complete course, of a standard of care treatment.

In some embodiments, a method herein comprises predicting an outcome of a cancer treatment. An outcome of a cancer treatment may be predicted when the presence of a structural variant described herein is detected. For example, an outcome of a cancer treatment that includes a gene-specific modulator and/or an oncogene-specific modulator may be predicted when the presence of a structural variant associated with the gene and/or oncogene is detected.

In some embodiments, a sample from a subject is obtained over a plurality of time points. A plurality of time points may include time point over a number of days, weeks, months, and/or years. In some embodiments, a disease state is monitored over a plurality of time points. For example, a method to detect the presence, absence, or amount of a structural variant described herein may be performed over a plurality of time points to monitor the status of a disease (e.g., a disease (e.g., cancer) associated with the structural variant detected). In some embodiments, minimal residual disease (MRD) is monitored in a subject. Minimal residual disease (MRD) generally refers to cancer cells remaining after treatment that often cannot be detected by standard scans (e.g., X-ray, mammogram, computerized tomography (CT) scan, bone scan, magnetic resonance imaging (MRI), positron emission tomography (PET) scan, ultrasound) or tests (blood test, tissue biopsy, needle biopsy, liquid biopsy, endoscopic exam). Such cells can have the potential to cause a relapse of cancer in a subject. In some embodiments, a method herein can include detecting a presence of minimal residual disease (MRD) in a subject when a structural variant described herein is present. In some embodiments, a method herein comprises detecting an absence of minimal residual disease (MRD) in a subject when a structural variant described herein is absent. In some embodiments, a method herein comprises detecting an amount of a structural variant described herein in a sample. A level of minimal residual disease (MRD) in a subject may be determined according to an amount of structural variant detected in a sample.

Treatment that Inhibits Polyadenosine Diphosphate-Ribose Polymerase (PARP) Enzyme.

In the methods provided herein, subjects having a structural variant associated with and/or adjacent to the HRR gene are selected for treatment and/or are treated with homologous recombination deficient direct therapy, such as an inhibitor of a polyadenosine diphosphate-ribose polymerase (PARP) enzyme.

An example of PARP presumed method of function, is reproduced below The description is from “Lord C J, Ashworth A. PARP inhibitors: Synthetic lethality in the clinic. Science. 2017; 355(6330):1152-1158. doi:10.1126/science.aam7344.” The focus on the discussion of any particular HRR gene is not meant to be limiting in any way to Applicant's invention, it is simply an example describing the method of how some HRR genes are thought to function and their possible roles in cancer.

“DNA damage and its repair or lack thereof is central to the induction of mutations, which drive the development of nearly all cancers. Healthy cells defend themselves against the deleterious effects of DNA damage through an inter-related series of molecular pathways, the DNA damage response (DDR), that recognize DNA damage, stall the cell cycle and mediate DNA repair, thus maintaining the integrity of the genome. Key to the DDR are the Poly(ADP-ribose) Polymerase 1 and 2 (PARP1 and PARP2) enzymes, DNA damage sensors and signal transducers that operate by synthesizing negatively charged, branched poly(ADP-ribose) (PAR) chains (PARylation) on target proteins as a form of post-translational modification. An understanding of the functions of PARP1 and PARP2 in the DDR drove long-standing efforts to develop small molecule PARP1/2 inhibitors (PARPi). The original rationale was that PARPi could sensitize tumor cells to conventional treatments that cause DNA damage, including multiple chemotherapy or radiotherapy approaches, which remain the backbone of treatment for most cancer patients. By inhibiting PARP-mediated repair of DNA lesions created by chemo- or radiotherapy, greater potency might be achieved. Carriers of deleterious mutations in the BRCA1 and BRCA2 genes have significantly elevated risks of developing breast, ovarian and other cancers. Both BRCA1 and BRCA2 proteins are critical to the repair of double strand DNA breaks by a process called homologous recombination repair (HRR), a form of DNA repair that uses a homologous DNA sequence to guide repair at the DSB. HRR is generally a “conservative” mechanism, in that it restores the original DNA sequence at the site of DNA damage. When cells become HRR deficient, whether driven by defects in BRCA1, BRCA2 or other pathway components, non-conservative forms of DNA repair predominate, such as Non-Homologous End Joining (NHEJ). These processes either fuse broken DNA ends at the DSBs without using a homologous DNA sequence to guide repair or fuse regions of DNA close the site of the DSB that exhibit short regions of DNA sequence homology, deleting the intervening DNA sequence. In 2005, two groups described the Synthetic Lethal (SL) interaction between PARP inhibition and BRCA1 or BRCA2 mutation, suggesting a novel strategy for treating patients with BRCA-mutant tumors. SL is a concept introduced nearly a century ago by geneticists to describe the situation whereby a defect in either one of two genes has little effect on the cell or organism but a combination of defects in both genes results in death. The demonstration that BRCA-mutant tumor cells were as much as 1,000 times more sensitive to PARPi than BRCA-wild type cells (depending on the PARPi used and the experimental formal) provided the impetus for PARPi to be tested in clinical trials as single agents. Originally it was proposed that the mechanism underlying the SL interaction was that PARP inhibition caused persistent SSBs which, when encountered by a replication fork sometimes resulted in collapse of the fork, potentially creating a DSB. However, this initial model has recently been modified as a result of data suggesting that some PARPi (especially rucaparib, olaparib, niraparib and talazoparib) “trap” PARP1 on DNA, preventing autoPARylation and PARP1 release from the site of damage and therefore interfering with the catalytic cycle of PARP1.”

In addition to BRCA1/2, it is believed that alterations in other HRR genes may confer sensitivity to HRD directed therapies, such as PARP inhibition. A treatment that utilizes homologous recombination deficient direct therapy, such as an inhibitor of a polyadenosine diphosphate-ribose polymerase (PARP) enzyme can provide better outcomes for subjects having cancer.

A treatment that homologous recombination deficient direct therapy, such as an inhibitor of a polyadenosine diphosphate-ribose polymerase (PARP) enzyme can include, but is not limited to, administering one or more agents selected from among atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A. Any agent or combination(s) thereof that acts as a homologous recombination deficient direct therapy, such as an inhibitor of a polyadenosine diphosphate-ribose polymerase (PARP) enzyme, can be administered in the methods provided herein. Non-limiting examples of an agent can include a small molecule, a nucleic acid (e.g., DNA, RNA such as mRNA or siRNA, PNA), and a protein, such as fusion protein, a modified PARP or PARP associated protein, or an antibody (including bispecific antibodies, diabodies, and the like) or an antigen-binding fragment thereof.

Knowing the structures and mechanisms of action of homologous recombination deficient direct therapy, such as an inhibitor of a polyadenosine diphosphate-ribose polymerase (PARP) enzyme and ligands/other components thereof, and given the knowledge regarding the structures and functions available agents that inhibit the pathway(s), the receptor, the ligands and/or other components, it is within the level of one of skill to design and produce additional agents that can act as an homologous recombination deficient direct therapy, such as an inhibitor of polyadenosine diphosphate-ribose polymerase (PARP) enzyme.

For example, when the agent is an antibody, such as a polyclonal antibody or a monoclonal antibody, it can be prepared using standard methods (see, e.g., Kohler et al., Nature 256:495-497 (1975); Kohler et al., Eur. J. Immunol. 6:511-519 (1976); and WO 02/46455). For example, to generate polyclonal antibodies, an immune response is elicited in a host animal, to an antigen of interest. Blood from the host animal is then collected and the serum fraction containing the secreted antibodies is separated from the cellular fraction, using methods known to those of skill in the art. To generate monoclonal antibodies, an animal is immunized by standard methods to produce antibody-secreting somatic cells. These cells then are removed from the immunized animal for fusion to myeloma cells. Somatic cells that can produce antibodies, particularly B cells, can be used for fusion with a myeloma cell line. These somatic cells can be derived from the lymph nodes, spleens and peripheral blood of primed animals. Specialized myeloma cell lines have been developed from lymphocytic tumors for use in hybridoma-producing fusion procedures (Kohler and Milstein, Eur. J. Immunol. 6:511-519 (1976); Shulman et al., Nature, 276:269-282 (1978); Volk et al., J. Virol., 42:220-227 (1982)). These cell lines have three useful properties. The first is they facilitate the selection of fused hybridomas from unfused and similarly indefinitely self-propagating myeloma cells by having enzyme deficiencies that render them incapable of growing in selective medium that support the growth of hybridomas. The second is they have the ability to produce antibodies and are incapable of producing endogenous light or heavy immunoglobulin chains. A third property is they efficiently fuse with other cells. Other methods for producing hybridomas and monoclonal antibodies are well known to those of skill in the art. It is routine to produce antibodies against any polypeptide, e.g., antigenic marker on an immune cell population.

Typically, monoclonal antibodies are developed in mice, rats or rabbits. The antibodies can be produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization can be cultured for a period of time (e.g., at least 24 hours) prior to their use as an immunogen. Cells can be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells can allow antigens to be better detected than ruptured cells by the immunized animal. Use of denaturing or harsh adjuvants, e.g., Freud's adjuvant, can rupture cells. The immunogen can be administered multiple times at periodic intervals such as, bi-weekly, or weekly, or can be administered in such a way as to maintain viability in the animal (e.g., in a tissue recombinant). Alternately, existing monoclonal antibodies and any other equivalent antibodies that are immunospecific for a desired pathogenic epitope can be sequenced and produced recombinantly by any means known in the art.

In aspects, an antibody can be sequenced, and the component polynucleotide sequences (or single sequence, in the case of ScFv) can then be cloned into a vector for expression or propagation. The polynucleotide sequence(s) encoding the antibody of interest can be maintained in a vector in a host cell and the host cell can then be expanded and frozen for future use. The polynucleotide sequence(s) of such antibodies can also be used for genetic manipulation to generate multispecific (e.g., bispecific, trispecific and tetraspecific) binding molecules as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody such as 1, 2, 3, 4, 5 or all 6 of the CDR sequences, while swapping the non-human remainder of the antibody with human antibody sequences.

Other proteins that are therapeutic agents, such as modified PARP associated ligands or peptides or other components that are part of, modulate or interact with PARP or PARP modulated pathway, can be produced by direct peptide synthesis, using, for example, well-known solid-phase techniques (see e.g., Stewart et al. (1969) Solid-Phase Peptide Synthesis, WH Freeman Co., San Francisco; Merrifield J (1963) J Am Chem Soc., 85:2149-2154). In vitro protein synthesis can be performed using manual techniques or by automation. Automated synthesis can be achieved, for example, using Applied Biosystems 431A Peptide Synthesizer (Perkin Elmer, Foster City CA) in accordance with the instructions provided by the manufacturer. Various fragments of a polypeptide can be chemically synthesized separately and combined using chemical methods. Such proteins also can be produced by recombinant means using well-known molecular biological methods of expressing proteins encoded by cloned DNA. In embodiments, the proteins can be mutated forms of a known therapeutic agent or can be mutated forms of PARP associated ligands or peptides or other components that are part of, modulate or interact with PARP or PARP modulated pathway Any method known in the art to effect mutation of any one or more amino acids in a target protein can be employed. Methods include standard site-directed or random mutagenesis of encoding nucleic acid molecules, or solid phase polypeptide synthesis methods. For example, nucleic acid molecules encoding a protein can be subjected to mutagenesis, such as random mutagenesis of the encoding nucleic acid, error-prone PCR, site-directed mutagenesis, overlap PCR, gene shuffling, or other recombinant methods. The nucleic acid encoding the polypeptides can then be introduced into a host cell to be expressed heterologously. In embodiments, the protein therapeutic agent can be a fusion protein or polypeptide. The fusion protein or polypeptide can be generated by direct chemical synthesis. The fusion protein or polypeptide also can be produced as a recombinant fusion polypeptide encoded by a nucleic acid sequence containing a coding sequence from one nucleic acid molecule and the coding sequence from another nucleic acid molecule in which the coding sequences are in the same reading frame such that when the fusion construct is transcribed and translated in a host cell, a fusion protein or polypeptide containing two or more different protein or polypeptide components is produced. The two different molecules can be adjacent in the construct or can be separated by a linker polypeptide that contains, e.g., 1, 2, 3, or more, but typically fewer than 10, 9, 8, 7, or 6 amino acids.

Typically, the agents (therapeutic agents) are administered in an amount that does not result in undesirable side effects of the subject (patient) being treated, or that minimizes or reduces the observed side effects and it is within the level of one of skill in the art to determine the appropriate dosages of these agents when administered singly, as a combination of two or more of the agent, or as a combination that includes all or part of a standard of care treatment regimen. Agents can be administered as a single dosage administration or as a multiple dosage administration. In certain aspects, an agent can be administered as a sustained release formulation. In aspects, the agents can be administered as an intravenous dose. The intravenous dose can be administered as a one-time treatment, or can be administered at intervals, such as, for example, once every 3 days, 4 days, 5 days, 6 days, 1 week, 2 weeks, 3 weeks, 1 month, 2 months, 3 months, or longer intervals. In aspects, the interval is 2 weeks. The dosage amount can range from about or 0.1 mg/kg to about or 100 mg/kg, such as, for example, about or 0.5 mg/kg to about or 50 mg/kg, about or 5 mg/kg to about or 50 mg/kg, about or 1 mg/kg to about or 20 mg/kg, about or 1 mg/kg to about or 100 mg/kg, about or 10 mg/kg to about or 80 mg/kg, or about or 50 mg/kg to about or 100 mg/kg or more; or at a dosage of about or 0.01 mg/m² to about or 800 mg/m² or more, such as for example, about or 0.01 mg/m², about or 0.1 mg/m², about or 0.5 mg/m², about or 1 mg/m², about or 5 mg/m², about or 10 mg/m², about or 15 mg/m², about or 20 mg/m², about or 25 mg/m², about or 30 mg/m², about or 35 mg/m², about or 40 mg/m², about or 45 mg/m², about or 50 mg/m², about or 100 mg/m², about or 150 mg/m², about or 200 mg/m², about or 250 mg/m², about or 300 mg/m², about or 400 mg/m², about or 500 mg/m², about or 600 mg/m² about or 700 mg/m².

Fusions (proximity or gene) are a new type of mutation that compromising the function of homologous recombination repair (HRR) In cancer cells. Small variants, such as point mutations and InDels are the mutation types known to compromise the function of homologous recombination repair (HRR) genes in cancer cells rendering them homologous recombination deficient (HRD). If a fusion (based on any method, such as but not limited to spatial proximity analysis) is identified associated with a HRR gene, patients will respond better to therapies that are known to be efficacious in the context of homologous recombination deficiency (HRD), including but not limited to PARP inhibitors or platinum-based therapies (Lise M. van Wijk, Andreea B. Nilas, Harry Vrieling & Maaike P. G. Vreeswijk (2022) RAD51 as a functional biomarker for homologous recombination deficiency in cancer: a promising addition to the HRD toolbox?, Expert Review of Molecular Diagnostics, 22:2, 185-199, DOI: 10.1080/14737159.2022.2020102).

Accordingly, using PARP inhibitors to counteract the effect of a fusion in or in proximity to an HRR gene will apply to all cancer types, and the evidence that it is “pan cancer” is shown in Table 8 where HRR fusions in multiple tumor types are described, with an emphasis on sarcomas. Essentially HRR gene rearrangements, in sarcomas, but also in pan-cancers, predicts response to HRD-directed therapies. The above embodiments are HRR gene-centric—they depend on finding a fusion of a gene whose protein function is in the HRR pathway.

Following are embodiments described that are gene-agnostic, overall signals from the spatial proximity analysis, such as, but not limited to HiC, data that can be indicative of HRD and predict response to HRD-directed therapies. For example, the total number of fusions are indicative of HRD, with a higher number of fusions being indicative of HRD, or indicative of an increased likelihood of HRD and predictive of response to therapies that are known to be efficacious in the context of HRD, including but not limited to PARP inhibitors or platinum-based therapies. A sample could be assigned a “score” that could be based on the number of fusions in a sample. For example from genome-wide HiC, a sample with 40 fusions could be a high HRD score, and one with 1 fusion is low a HRD score. This could also be detected in the context of capture-HiC or other targeted techniques. The main idea would be that the “burden” of fusions is indicative of HRD. Fusion-based HRD detection could also be carried out using genome-wide assays such as genome-wide spatial proximity analyses (or in the context pf targeted assays such as targeted spatial proximity analysis (such as, but not limited to, Capture-Hic or other targeted techniques). The discovery that a high number of fusion events can be used to indicate PARP inhibitor or platinum-based therapies apply to ALL cancer types. Essentially fusion burden and an HRD score based off fusion burden, in sarcomas, but also in pan-cancers, predicts response to HRD-directed therapies, irrespective of the detection of a HRR gene rearrangement. Fusion burden is the consequence of ANY event that affects homologous recombination repair and leads to genomic instability.

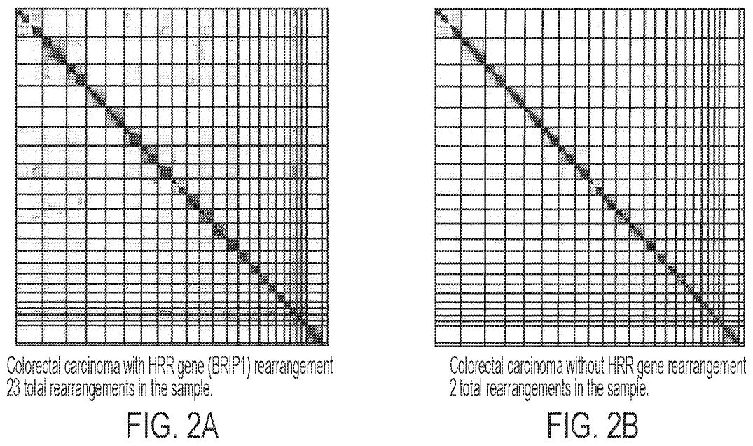

Applicants have discovered that HRR gene rearrangements impact several HRR genes (see Table 1 and Table 8) and are found in several cancer types (See Table 8), indicating that HRR gene rearrangements may be a “pan-cancer” biomarker for HRD and indicate which patients will respond favorably to HRD directed therapies. The data also indicate that the prevalence of HRR alterations is higher than currently known, given that rearrangements of HRR genes was an alteration type not previously considered to after function of HRR genes. Evidence that HRR gene rearrangements lead to HRD can be observed by the degree of genomic instability in a sample, measured in the nucleic acid analyses described herein as the total number of genomic rearrangements in a sample, with a higher number of rearrangements indicative of HRD. Using the spatial-proximal contiguity analyses described herein, a common feature of samples with HRR gene rearrangements is a higher total number of genomic rearrangements than samples without HRR gene rearrangements, indicating HRD. To illustrate this point using matched analyses of the same tumor type with and without HRR gene alterations, we show significantly more intra- and inter-chromosomal rearrangements in samples with HRR gene rearrangements compared to those without (See FIGS. 2-5). It is therefore anticipated that detection of HRR gene rearrangements, such as by using a spatial proximal contiguity analysis, may be indicative of HRD correlates with response to HRD directed therapies.

In embodiments, a higher number of rearrangements (also described as an increased number of gene alterations and/or structural variants) means any number of rearrangements over 5, over 6, over 7, over 8, over 9, over 10, over 15, or over 20. In preferred embodiments, the number of rearrangements indicative of response to HRD directed therapies is 10 or more.

In addition, it is anticipated that there are gene-agnostic, overall signals from a spatial proximal contiguity analysis (Hi-C) that would be indicative of HRD and predict response to HRD-directed therapies. For example, even in the absence of detecting a gene rearrangement involving a known HRR gene, the total number of rearrangements detected could be indicative of HRD, with a higher number of fusions being indicative of HRD, or indicative of an increased likelihood of HRD and therefore predictive of response to therapies that are known to be efficacious in the context of HRD, including but not limited to PARP inhibitors or platinum-based therapies. Rearrangement-based HRD detection, such as by using a spatial proximal contiguity analysis, could be carried out using genome-wide spatial proximal contiguity analysis (Hi-C), or in the context of targeted assays such as targeted spatial proximal contiguity analysis (Capture-HiC or other targeted techniques).

It is expected that the total number of fusions found in a multitude of tumor types, including especially sarcomas, will increase when there is a disruption (such as, but not limited to a structural variant breakpoint) near or in an HRR gene.

### EXAMPLES

The examples set forth below illustrate certain implementations and do not limit the technology.

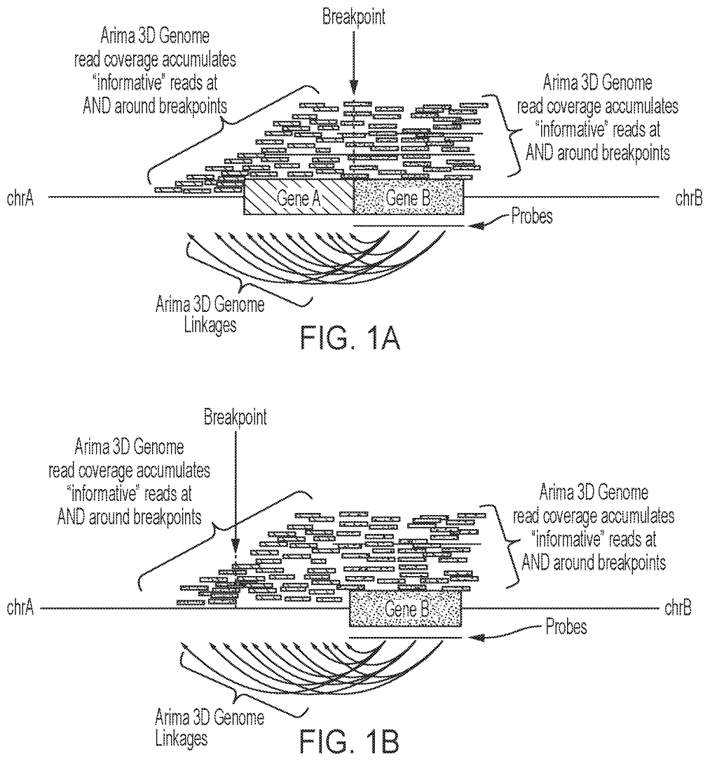

FIG. 1A shows a schematic of Capture-HiC data using target enrichment probes targeted to cancer genes, in order to identify a SV that results in a gene fusion. The schematic shows a SV between hypothetical chromosome A and hypothetical chromosome B, which creates a gene fusion between Gene A (on chromosome A) and Gene B (on chromosome B). The breakpoint is located in the center, where Gene A is fused to Gene B. The horizontal bar below Gene B depicts the targeting of probes to enrich for Gene B during the Capture-HiC workflow. The “arcs with arrows” at the bottom depict the concept that a captured HiC fragment containing Gene B may also contain a fragment from Gene A, or the genetic locus around Gene A, due to the nature of capturing 3D spatial proximity of DNA. This concept is portrayed in the figure as “3D Genome Linkages”—meaning fragments that are linked between Gene B and Gene A due to spatial proximity. There would also likely be a fragment between Gene B and Gene A or the locus around Gene B, but those are not depicted as they are not necessarily informative to detect a structural variant (SV) between chrA and chrB. Above the chromosome depicts dark gray and light gray sequence reads from this hypothetical Capture-HiC experiment. Dark gray fragments are derived from chrB and light gray fragments are derived from chrA. The intended depiction here is that each dark gray fragment (or sequence read) is linked to a light gray fragment and thus informative to detect an SV between chrA and chrB. An entirely dark gray fragment can be linked to an entirely light gray fragment, and still be informative despite neither fragment containing the breakpoint. Also depicted here is the notion that some sequence reads will contain the actual breakpoint, indicated by a black tick mark. Lastly, it is intentionally depicted here that the read coverage of reads linked to Gene B get lesser as one moves further away along the genome from Gene B. This is to reflect the property of the 3D genome that the spatial proximity between any two points along the genome is higher when they are linearly proximal, and further when they are linearly distal along a chromosome.

FIG. 1B shows a schematic of Capture-HiC data using target enrichment probes targeted to cancer genes, in order to identify a SV that results in a breakpoint outside of the targeted gene body. Shown here is a schematic similar to FIG. 1, but with the following differences. First, the breakpoint here is outside of the targeted gene body. Shown here the breakpoint does not lie within a gene, but the same principle would be true if the breakpoint lied within a non-targeted gene as the core concept of this figure is to illustrate the detection of SVs where the breakpoints lie outside of any targeted gene (or any targeted sequence/region). Because the breakpoint is outside of Gene B, the dark gray fragments/reads directly above the Gene B icon can be linked to either light gray fragments from chrA, or, dark gray fragments from chrB but outside of chrB between Gene B and chrA. Those reads where both linked fragments are dark gray are not particularly informative to SV and breakpoint detection, only those between gene B and chrA. Also note that it is intentionally depicted that some reads linked to Gene B are both dark gray and light gray and contain the breakpoint. This is intended to show that the sequence fragment containing the breakpoint may spatially interact with sequence elements from the targeted Gene B, making it possible for targeted HiC data to detect not only the SVs (light gray to dark gray linkages), but also the breakpoint itself (dark gray to light gray/dark gray linkages). The number of breakpoints containing fragments and the total number of linkages between Gene B aid chrA would be influenced by the linear distance between the breakpoint and the enriched gene due to the property of the 3D genome that the spatial proximity between any two points along the genome is higher when they are linearly proximal, and further when they are linearly distal along a chromosome.

### Example 1: Identification of Structural Variants in Cancer Samples

In this Example, the identification of structural variants in cancer samples is described.

### HiC for FFPE

For FFPE samples, 1-10 FFPE sections of 5-10 μm thickness were subject to a HiC protocol for FFPE tissues (Arima Genomics, San Diego, CA). The FFPE samples were deparaffinized and rehydrated using one incubation with Xylene, one incubation with 100% ethanol, and one incubation with water. Following the water incubation, the deparaffinized and rehydrated tissue was incubated in Lysis Buffer (formulation below in Table 1) on ice for 20 min.

TABLE 1
Lysis Buffer

| | Stock | | | Final | | Master |
|---|---|---|---|---|---|---|
| Reagent | Conc. | Units | μL/rxn | Conc. | Units | Mix |
| Tris-HCl | 1000 | mM | 1.667 | 8.333 | mM | 62.333 |
| pH 8.0 | | | | | | |
| NaCl | 1000 | mM | 1.667 | 8.333 | mM | 62.333 |
| IGEPAL | 10 | % | 3.333 | 0.167 | % | 124.667 |
| Protease | 100 | % | 33.333 | 16.667 | % | 1246.667 |
| Inhibitor | | | | | | |
| Cocktail | | | | | | |
| DI Water | | | 160.00 | | | 5984.000 |
| | | Total/rxn | 200.00 | μL/rxn | | 7480 |

Following lysis incubation, samples were pelleted, decanted, and resuspended in 20 μl of 1× Tris Buffer pH 7.4.

Then, 24 μl of Conditioning Solution (formulation below in Table 2) was added and the samples were incubated at 74° C. for 40 min.

TABLE 2
Conditioning Solution

| | Stock | | | Final | | Master |
|---|---|---|---|---|---|---|
| Reagent | Conc. | Units | μL/rxn | Conc. | Units | Mix |
| SDS | 20 | % | 1.104 | 0.920 | % | 41.290 |
| DI Water | | | 22.896 | | | 856.310 |
| | | Total/rxn | 24.000 | μL/rxn | | 897.6 |

20 μl of Stop Solution 2 (10.71% TritonX-100) was then added and the samples were incubated at 37° C. for 15 min.

After incubation in the Stop Solution, 12 μl of a Digestion Master Mix (formulation below in Table 3) was added and the samples were incubated for 1 hr at 37° C., followed by 20 min at 62° C.

TABLE 3
Digestion Master Mix

| | Stock | | | Final | | Master |
|---|---|---|---|---|---|---|
| Reagent | Conc. | Units | μL/rxn | Conc. | Units | Mix |
| NEB3.1 | 10 | x | 7.000 | | | 261.800 |
| DpnlI | 50 | U/ μL | 1 | | | 37.400 |
| HinfI | 50 | U/ μL | 4 | | | 149.6000 |
| | | Total/rxn | 12.000 | μL/rxn | | 448.8 |

Then, 16 μl of a Fill-in Master Mix (formulation below in Table 4) was added and the samples were incubated for 45 min at 23° C. (room temperature).

TABLE 4
Fill-In Master Mix

| | Stock | | | Final | | Master |
|---|---|---|---|---|---|---|
| Reagent | Conc. | Units | μL/rxn | Conc. | Units | Mix |
| dCTP | 10 | mM | 0.281 | 0.176 | mM | 10.509 |
| dGTP | 10 | mM | 0.281 | 0.176 | mM | 10.509 |
| dTTP | 10 | mM | 0.281 | 0.176 | mM | 10.509 |
| Biotin-dATP | 0.4 | mM | 7.013 | 0.175 | mM | 262.286 |
| 1X NEB3.1 | 1 | X | 4.144 | 0.259 | X | 154.986 |
| Klenow | 5 | U/ μL | 4.000 | 1.250 | U/ μL | 149.600 |
| | | Total/rxn | 16.000 | μL/rxn | | 598.4 |

82 μl of a Ligation Master Mix (formulation below in Table 5) was then added and the samples were incubated overnight at 23° C. (room temperature).

TABLE 5
Ligation Master Mix

| | Stock | | | Final | | Master |
|---|---|---|---|---|---|---|
| Reagent | Conc. | Units | μL/rxn | Conc. | Units | Mix |
| 10% TritonX- | 10 | % | 13.580 | 1.656 | % | 507.892 |
| 100 | | | | | | |
| BSA | 100 | X | 1.650 | 2.012 | X | 61.710 |
| Ligase | 10 | X | 16.500 | 2.012 | X | 617.100 |
| Buffer | | | | | | |
| T4 DNA | | | 12.00 | | | 448.800 |
| Ligase | | | | | | |
| DI Water | | | 38.270 | | | 1431.298 |
| | | Total/rxn | 82.000 | μL/rxn | | 3066.8 |

Following the ligation incubation, 16.6 μl of 5 M NaCl was added and the samples were incubated overnight at 65° C.

Then, 35.5 μl of a Reverse Crosslinking Master Mix (formulation below in Table 6) was added and the samples were incubated overnight at 55° C.

TABLE 6
Reverse Crosslinking Master Mix

| | Stock | | | Final | | Master |
|---|---|---|---|---|---|---|
| Reagent | Conc. | Units | μL/rxn | Conc. | Units | Mix |
| SDS | 20 | % | 10.500 | 2.561 | % | 261.800 |
| Proteinase K | | | 25.000 | | | 935.000 |
| | | Total/rxn | 35.000 | μL/rxn | | 1327.7 |

Following the reverse crosslinking incubation, DNA was purified using SPRI beads and then sonicated/sheared. DNA was size selected for fragments 200-600 bp in length using SPRI beads. Biotinylated DNA was enriched using Streptavidin beads, and on-bead DNA fragments were converted into adapter ligated Illumina sequencing libraries using reagents from the SWIFT ACCEL-NGS 2S Pius DNA Library Kit (Swift Biosciences/IDT).

Then, adapter ligated and bead-bound DNA was PCR amplified using reagents from KAPA, and the resulting PCR-amplified DNA was purified using SPRI beads. For samples subject to Capture-HiC, sufficient PCR cycles were used in order to obtain at least 500 ng (optimally 1500 ng) of DNA (the minimum amount of DNA used for probe hybridization in the Capture-HiC protocol). HiC libraries were subject to shallow sequencing 00 on an Illumina MINISEQ. HiC libraries were subject to deep NGS on either Illumina HISEQ or NOVASEQ instruments.

### HiC for Blood

The HiC protocol for blood (Arima Genomics. San Diego, CA) matches that of FFPE protocol described above, except for the following differences.

Blood samples are not already fixed and then are not paraffin embedded. Therefore, the first step for blood is to crosslink blood cells using 2% formaldehyde for 10 min, quench crosslinking using a final concentration of 125 mM Glycine, and then begin HiC with the Lysis Step (see above).

The blood protocol differs from FFPE in the Conditioning Solution step, where Conditioning Solution for blood is added at 62° C. for 10 min. The blood protocol also differs from FFPE in the Ligation step, where Ligation reaction is 15 min instead of overnight. The blood protocol also differs from FFPE after Ligation but before DNA purification, in that a single Reverse Crosslinking master mix containing Proteinase K. NaCl, and SDS is added to the sample and it is incubated at 55° C. for 30 min, then 68° C. for 90 min, and then purified using SPRI beads.

The remainder of the protocol, including DNA shearing, size selection, library prep, PCR and Capture-HiC (below) is the same between blood and FFPE.

### Capture-HiC

First, 1500 ng of amplified HiC library was “pre-cleared” in order to remove residual biotinylated DNA. This was done by negative selection—the 1500 ng of amplified HiC library was combined with streptavidin beads, and the unbound DNA fraction was carried forward and the bound fraction was discarded.

The now pre-cleared amplified HiC library was then subject to Capture Enrichment, consisting of a) hybridization, b) capture; and c) amplification; according to the Agilent SURESELECT XTHS reagents and standard protocol. Capture targets/probes were custom-designed by Arima, using the Agilent SUREDESIGN software suite (details below). Following Capture Enrichment, Capture-HiC libraries were shallow sequenced on a MINISEQ or more deeply sequenced on an Illumina HISEQ.

### Capture Probe Design

A list of unique genes was compiled from the following sources:
NYU GenomePACT Panel
NYU Fusion SEQ'r Panel
ArcherDx VaiantPlex Myeloid Panel
ArcherDx Pan Heme Panel
Stanford STAMP Heme Panel
ArcherDx Pan Solid Tumor
ArcherDx VariantPlex Solid Tumor
Childrens' Hospital of Philadelphia (CHOP) Comprehensive Tumor and Fusion Panel
Agilent All-in-One Solid Tumor Panel
Agilent ClearSeq Comprehensive Cancer Panel
Foundation Medicine Foundation One CDx Panel
Stanford STAMP Solid Tumor Panel
Stanford STAMP Fusion Panel

These genes were then cross-referenced to the Ensembl data base, with 885 total genes collected (see Table 1 below). The exon coordinates were then located for all 885 genes, as well as the HiC restriction enzyme cut sites (Arima Genomics, San Diego, CA) within and directly flanking the exons. To define the target capture regions, the sequences within 350 bp from restriction enzyme cut sites were identified. For cut sites flanking the exons, the “inward” 350 bp (the 350 bp in the direction of the exon) was targeted. For this probe design, the cut sites were: \hat{ }GATC and G\hat{ }ANTC (where A is the cut site on the positive strand, and “N” can be any of the 4 genomic bases, A, C, G, T). Collectively, this approach identified a set of coordinates in and around exons of genes of interest. These coordinates were then uploaded into the Agilent SUREDESIGN™ Software Suite for the design of individual probe sequences. Probe design was carried out using some custom parameters, including 1× tiling density, moderate stringency repeat masking, and optimized performance boosting. The probes were designed against the HG38 human reference genome. The total size of the target region was 12.075 Mb and following probe design 92.79449% (11.483 Mb) was covered by probes. In total, 335,242 probes were designed.

TABLE 7
Oncopanel genes

ABCB1
ABCC2
ABL1
ABL2
ABRAXAS1
ACTG1
ACVR1
ACVR1B
ACVR2A
ADAMTS20
ADGRA2
ADGRB3
ADGRF5
ADGRL3
AFDN
AFF1
AFF3
AICDA
AKAP9
AKT1
AKT2
AKT3
ALK
ALOX12B
AMER1
ANKRD24
ANKRD26
APC
APLNR
AR
ARAF
ARFGAP3
ARFRP1
ARHGAP26
ARHGAP6
ARID1A
ARID1B
ARID2
ARNT
ASB13
ASH1L
ASPSCR1
ASXL1
ATF1
ATM
ATR
ATRX
AURKA
AURKB
AURKC
AUTS2
AXIN1
AXL
B2M
BAP1
BARD1
BATF3
BAX
BCL10
BCL11A
BCL11B
BCL2
BCL2A1
BCL2L1
BCL2L2
BCL3
BCL6
BCL9
BCOR
BCORL1
BCR
BEND2
BIRC2
BIRC3
BIRC5
BLM
BLNK
BMF
BMP7
BMPR1A
BOD1L1
BRAF
BRCA1
BRCA2
BRD3
BRD4
BRINP3
BRIP1
BTG1
BTK
BUB1B
CACNA1E
CALR
CAMTA1
CARD11
CASP8
CBFA2T3
CBFB
CBL
CBLB
CBLC
CCDC170
CCDC50
CCN6
CCNB3
CCND1
CCND2
CCND3
CCNE1
CCR4
CD22
CD274
CD28
CD44
CD58
CD70
CD74
CD79A
CD79B
CD83
CDA
CDC25A
CDC25C
CDC73
CDH1
CDH11
CDH2
CDH20
CDH23
CDH5
CDK12
CDK4
CDK6
CDK8
CDKN1A
CDKN1B
CDKN2A
CDKN2B
CDKN2C
CEBPA
CEBPD
CEBPE
CEBPG
CHD1
CHD2
CHD4
CHD5
CHD7
CHEK1
CHEK2
CHIC2
CIC
CIITA
CILK1
CKS1B
CMPK1
COL1A1
CRBN
CREB1
CREB3L2
CREBBP
CRKL
CRLF2
CRTC1
CSF1
CSF1R
CSF3R
CSMD3
CSNK2B
CTCF
CTDNEP1
CTLA4
CTNNA1
CTNNB1
CUL3
CUL4A
CUX1
CXCR4
CXXC5
CYB5R2
CYLD
CYP17A1
CYP19A1
CYP2A6
CYP2B6
CYP2C19
CYP2C9
CYP2D6
DAXX
DCC
DCK
DDB2
DDIT3
DDR1
DDR2
DDX3X
DDX41
DEK
DENND3
DHX15
DICER1
DIS3
DLEU1
DNAH9
DNAJB1
DNM2
DNMT3A
DNMT3B
DNTT
DOT1L
DPH3
DPYD
DROSHA
DST
DUSP22
E2F2
EBF1
EED
EGF
EGFR
EGR1
EIF4A1
EML4
EMSY
ENTPD1
EP300
EP400
EPC1
EPCAM
EPHA2
EPHA3
EPHA5
EPHA7
EPHB1
EPHB4
EPHB6
EPOR
ERBB2
ERBB3
ERBB4
ERCC1
ERCC2
ERCC3
ERCC4
ERCC5
ERG
ERRFI1
ESR1
ESR2
ESRRA
ETNK1
ETS1
ETV1
ETV4
ETV5
ETV6
EWSR1
EXOC2
EXT1
EXT2
EZH1
EZH2
EZR
FAM216A
FANCA
FANCC
FANCD2
FANCE
FANCF
FANCG
FANCL
FAS
FBXW4
FBXW7
FGF1
FGF10
FGF12
FGF14
FGF19
FGF23
FGF3
FGF4
FGF6
FGFR1
FGFR2
FGFR3
FGFR4
FGR
FH
FIP1L1
FLCN
FLI1
FLT1
FLT3
FLT4
FN1
FOS
FOSB
FOXA1
FOXL2
FOXO1
FOXO3
FOXO4
FOXP1
FOXP4
FOXR2
FSTL5
FUBP1
FUS
FUT8
FYN
FZR1
G6PD
GABRA6
GATA1
GATA2
GATA3
GATA6
GDNF
GID4
GLI1
GLIS2
GNA11
GNA13
GNAI3
GNAQ
GNAS
GNB1
GPS2
GRB7
GRIN2A
GRM3
GRM8
GSK3B
GSTP1
GUCY1A2
H1-2
H1-3
H1-4
H1-5
H2AC6
H3-3A
H3-3B
H3C14
H3C2
HCAR1
HDAC1
HGF
HIF1A
HLF
HMGA2
HNF1A
HNRNPK
HOOK3
HOXA10
HOXA9
HOXB13
HRAS
HSD3B1
HSP90AA1
HSP90AB1
ID3
ID4
IDH1
IDH2
IGF1R
IGF2
IGF2R
IGHA1
IGHA2
IGHG1
IGHG2
IGHG3
IGHG4
IGHJ1
IGHJ2
IGHJ3
IGHJ4
IGHJ5
IGHJ6
IGHM
IKBKB
IKBKE
IKZF1
IKZF2
IKZF3
IL16
IL2
IL21R
IL2RA
IL2RB
IL2RG
IL3
IL3RA
IL6ST
IL7R
ING4
INHBA
INPP4B
INSR
IRAG2
RF2
IRF4
IRF8
IRS2
ITGA10
ITGA9
ITGB2
ITGB3
ITK
ITPKB
JAK1
JAK2
JAK3
JARID2
JAZF1
JMJD1C
JUN
KAT6A
KAT6B
KDM5A
KDM5C
KDM6A
KDR
KEAP1
KEL
KIT
KLF2
KLF6
KLHL6
KMT2A
KMT2B
KMT2C
KMT2D
KNL1
KRAS
LAMA2
LAMP1
LCK
LIFR
LIMD1
LMO1
LMO2
LPP
LRP1B
LTF
LTK
LUC7L2
LYL1
LYN
LZTR1
LZTS1
MAF
MAFB
MAGEA1
MAGI1
MAL
MALT1
MAML2
MAML3
MAMLD1
MAP2K1
MAP2K2
MAP2K4
MAP3K1
MAP3K13
MAP3K7
MAPK1
MAPK8
MARK1
MARK4
MAST1
MAST2
MBD1
MBTD1
MCL1
MDM2
MDM4
MEAF6
MECOM
MED12
MED13
MEF2B
MEN1
MERTK
MET
MITF
MKNK1
MLC1
MLF1
MLH1
MLH3
MLLT1
MLLT10
MME
MMP2
MN1
MNX1
MPL
MRE11
MRTFA
MRTFB
MSH2
MSH3
MSH6
MSMB
MST1R
MTAP
MTOR
MTR
MTRR
MUC1
MUSK
MUTYH
MYB
MYBL1
MYC
MYCL
MYCN
MYD88
MYH11
MYH9
MYOD1
NAB2
NBN
NCOA1
NCOA2
NCOA3
NCOA4
NCOR2
NEK6
NELL2
NF1
NF2
NFATC2
NFE2L2
NFIB
NFKB1
NFKB2
NFKBIA
NFKBIE
NIN
NKX2-1
NLRP1
NME1
NOTCH1
NOTCH2
NOTCH3
NOTCH4
NPM1
NR4A3
NRAS
NRG1
NSD1
NSD2
NSD3
NT5C2
NTRK1
NTRK2
NTRK3
NUMA1
NUMBL
NUP214
NUP93
NUP98
NUTM1
NUTM2A
OGA
P2RY8
PAG1
PAICS
PAK3
PALB2
PARP1
PARP2
PARP3
PAX3
PAX5
PAX7
PAX8
PBRM1
PBX1
PCBP1
PCDHAC2
PCLAF
PDCD1
PDCD1LG2
PDE4DIP
PDGFB
PDGFD
PDGFRA
PDGFRB
PDK1
PER1
PGAP3
PHF1
PHF6
PHKB
PHLPP2
PHOX2B
PICALM
PIGA
PIK3C2B
PIK3C2G
PIK3C3
PIK3CA
PIK3CB
PIK3CD
PIK3CG
PIK3R1
PIK3R2
PIM1
PIM2
PKD1L2
PKHD1
PKN1
PLAG1
PLCG1
PLCG2
PLEKHG5
PLEKHS1
PML
PMS1
PMS2
POLD1
POLE
POT1
POU5F1
PPARG
PPAT
PPM1D
PPP2R1A
PPP2R2A
PPP6C
PRCC
PRDM1
PRDM10
PRDM16
PREX2
PRKACA
PRKACB
PRKAR1A
PRKAR2B
PRKCA
PRKCB
PRKCD
PRKCI
PRKD1
PRKD2
PRKD3
PRKDC
PRPF8
PSIP1
PSMB1
PSMB2
PSMB5
PSMD1
PSMD2
PTCH1
PTEN
PTGS2
PTK2B
PTPN1
PTPN11
PTPRD
PTPRO
PTPRT
PYCR1
QKI
RAB29
RAC1
RAD21
RAD50
RAD51
RAD51B
RAD51C
RAD51D
RAD52
RAD54L
RAF1
RAG1
RAG2
RALGDS
RANBP1
RARA
RARB
RARG
RB1
RBBP6
RBM10
RBM15
RECQL4
REL
RELA
RET
RHEB
RHOA
RHOH
RICTOR
RIT1
RNASEL
RNF2
RNF213
RNF43
ROS1
RPL22
RPN1
RPS14
RPS15
RPS6KA2
RPS6KB1
RPTOR
RRM1
RSPO2
RSPO3
RUNX1
RUNX1T1
RXRA
RXRB
RXRG
S1PR2
SAMD9
SBDS
SDC4
SDHA
SDHB
SDHC
SDHD
SEMA6A
SERPINA9
SETBP1
SETD2
SETD5
SF3B1
SGK1
SH2B3
SH2D1A
SH3BP5
SHH
SHOC2
SLC22A1
SLC22A2
SLC29A1
SLC31A1
SLC34A2
SLC45A3
SLCO1B1
SMAD2
SMAD4
SMARCA4
SMARCB1
SMARCE1
SMC1A
SMC3
SMO
SMUG1
SNCAIP
SNX31
SOCS1
SOCS3
SOS1
SOX10
SOX11
SOX2
SOX9
SP140
SPEN
SPI1
SPOP
SPRED1
SPTA1
SRC
SRSF2
SS18
SS18L1
SSX1
SSX2
SSX4
STAG2
STAT1
STAT3
STAT4
STAT5B
STAT6
STIL
STK11
STK36
STRBP
STX11
SUFU
SUZ12
SYK
SYNE1
SYT1
TAF1
TAF15
TAF1L
TAL1
TAS2R38
TBX22
TBX3
TCF12
TCF3
TCF7L1
TCF7L2
TCL1A
TEK
TENT5C
TERC
TERT
TET1
TET2
TET3
TFE3
TFEB
TFG
TGFB1
TGFBR2
TGFBR3
TGM7
THADA
THBS1
TIMP3
TIPARP
TLR2
TLR4
TLX1
TLX3
TMEM216
TMPRSS2
TNFAIP3
TNFRSF13B
TNFRSF14
TNFRSF1A
TNFRSF1B
TNFSF4
TNK2
TOP1
TP53
TP63
TPM3
TPR
TRAF3
TRIM24
TRIM33
TRIP11
TRRAP
TSC1
TSC2
TSHR
TSLP
TYK2
TYRO3
U2AF1
U2AF2
UBR5
UGT1A1
UMODL1
USP6
USP9X
VAV1
VEGFA
VGLL2
VGLL3
VHL
WAS
WRN
WT1
WWTR1
XPA
XPC
XPO1
XRCC2
YAP1
YES1
YWHAE
ZCCHC7
ZMYM2
ZMYM3
ZNF217
ZNF384
ZNF521
ZNF703
ZRSR2
ZSWIM4

### HiC Data Analysis

To identify structural variants, raw HiC read-pairs were mapped to the human reference (hg38) and deduplicated. Mapped and deduplicated read pairs were then analyzed using the HiC-BREAKFINDER software (Dixon, Nature Genetics, 2018) to call structural variants.

For data visualization, HiC read-pairs were analyzed using the JUICER software, which outputs a “.hic” file that can be uploaded into the desktop JUICEBOX software for visualization of HiC heatmaps. Visual inspection, along with the structural variant calls from HiC-BREAKFINDER, were used to approximate the structural variant breakpoints from HiC analysis.

FIG. 2A shows a genome-wide Hi-C analysis and heatmap of spatial proximal interactions between all chromosomes in the genome, and numerous inter-chromosomal rearrangements between different chromosomes in a driver-negative colorectal carcinoma tumor. This tumor had a detected HRR gene rearrangement involving the HRR gene BRIP1, and 23 total rearrangements in the sample. FIG. 2B shows a genome-wide Hi-C analysis and heatmap of spatial proximal interactions between all chromosomes in the genome in a driver-negative colorectal carcinoma tumor. This tumor did not have a detected HRR gene, and 2 total rearrangements in the sample.

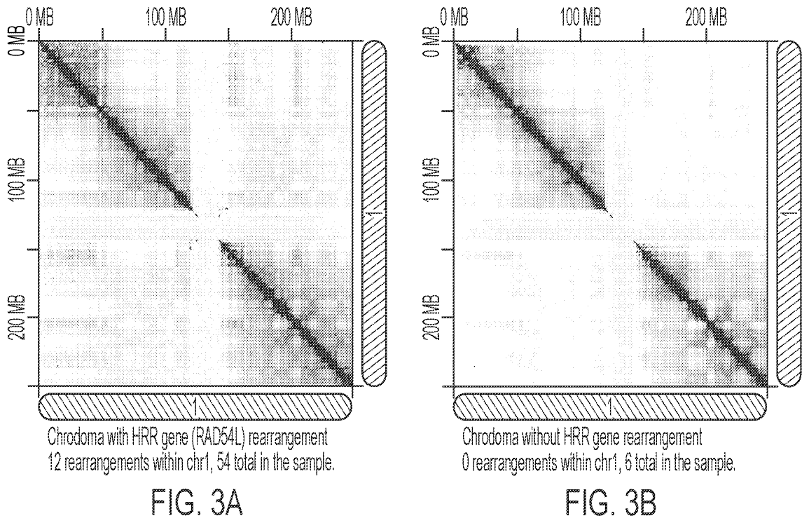

FIG. 3A shows a genome-wide Hi-C analysis and heatmap of spatial proximal interactions within chr1 and 12 intra-chromosomal rearrangements within chr1 in a driver-negative Chordoma tumor. This tumor had a detected HRR gene rearrangement involving the HRR gene RAD54L, and 54 total rearrangements in the sample. FIG. 3B shows a genome-wide Hi-C analysis and heatmap of spatial proximal interactions within chr1 and 0 intra-chromosomal rearrangements within chr1 in a driver-negative Chordoma tumor. This tumor did not have a detected HRR gene rearrangement, and 6 total rearrangements in the sample.

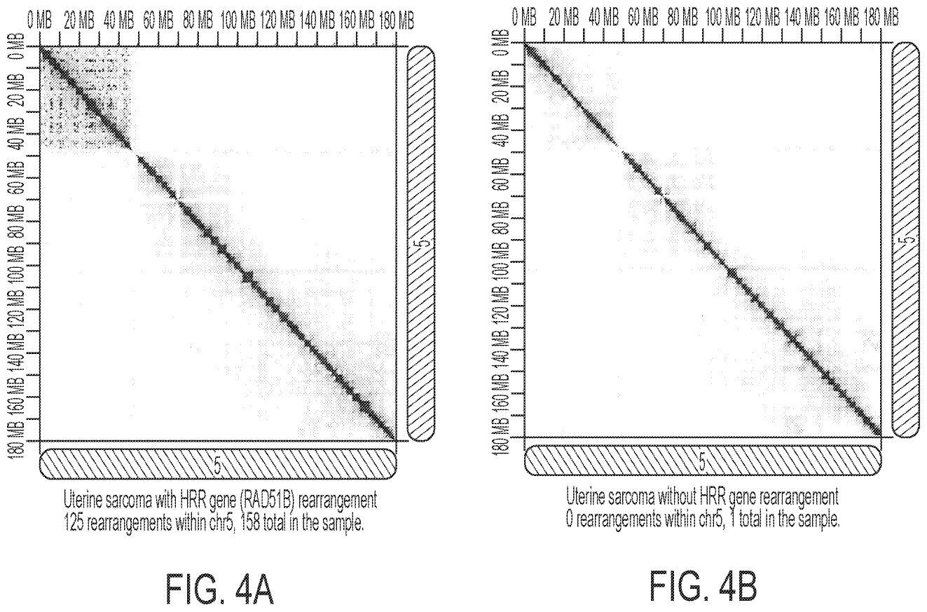

FIG. 4A shows a genome-wide Hi-C analysis and heatmap of spatial proximal interactions within chr5 and 125 intra-chromosomal rearrangements within chr5 in a driver-negative uterine sarcoma tumor. This tumor had a detected HRR gene rearrangement involving the HRR gene RAD51B, and 158 total rearrangements in the sample. FIG. 4B shows a genome-wide Hi-C analysis and heatmap of spatial proximal interactions within chr5 and 0 intra-chromosomal rearrangements within chr5 in a driver-negative uterine sarcoma tumor. This tumor did not have a detected HRR gene rearrangement, and 1 total rearrangement in the sample.

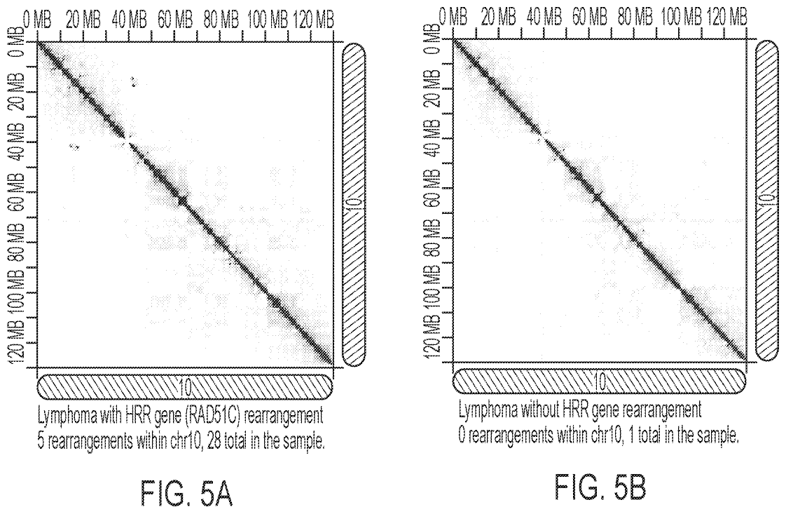

FIG. 5A shows a genome-wide Hi-C analysis and heatmap of spatial proximal interactions within chr10 and 5 intra-chromosomal rearrangements within chr10 in a driver-negative lymphoma tumor. This tumor had a detected HRR gene rearrangement involving the HRR gene RAD51C, and 28 total rearrangements in the sample. FIG. 5B shows a genome-wide Hi-C analysis and heatmap of spatial proximal interactions within chr10 and 0 intra-chromosomal rearrangements within chr10 in a driver-negative lymphoma tumor. This tumor did not have a detected HRR gene rearrangement, and 1 total rearrangement in the sample.

### Structural Variant Identified

Table 8 (encompassing all subtables) below shows the structural variants associated with and/or adjacent to the HRR genes of Table 1 that was identified by methods described herein. Certain samples were classified as having undiagnosed tumors/cancers with no clear with no known tumor driver (e.g., cancer gene/oncogene) as assessed by standard cytogenetic/molecular testing (i.e., chromosomal karyotyping, a FISH panel, DNA microarray, and/or a cancer next generation sequencing (NGS) panel).

TABLE 8.1

| Row | | | | | |
|---|---|---|---|---|---|
| 1 | VARIANT | 1 | 2 | 3 | 4 |
| | ID | | | | |
| 2 | SAMPLE | S1 | S2 | S3 | S4 |
| | NUMBER | | | | |
| 3 | Tumor type | Colorectal | Chordoma (PDx | Chordoma | Undifferentiated |
| | | Carcinoma | model) | | Uterine Sarcoma |
| | | | | | (UUS) - Uterine |
| 4 | Partner 1 | break in | break in | break in | break in |
| | type | PAN3 | FAM157C | NR_110931 | RAD51B |
| 5 | Approx. | chr13: 28,211,001- | chr16: 90, 100,001- | chr16: 89,460,001- | chr14: 68,678,001- |
| | breakpoint | 28,212,000 | 90,110,000 | 89,470,000 | 68,679,000 |
| | coordinate | | | | |
| | window 1A | | | | |
| 6 | Approx. | chr13: 28,210,001- | chr16: 90,090,001- | chr16: 89,450,001- | chr14: 68,676,001- |
| | breakpoint | 28,213,000 | 90,120,000 | 89,480,000 | 68,681,000 |
| | coordinate | | | | |
| | window 1B | | | | |
| 7 | Relevant | FLT3 | FANCA | FANCA | RAD51B |
| | cancer | | | | |
| | gene(s) | | | | |
| 8 | Gene 5′ | chr13: 28,100,576 | chr16: 89,816,647 | chr16: 89,816,647 | chr14: 67,865,032 |
| 9 | Gene 3′ | chr13: 28,003,274 | chr16: 89,737,549 | chr16: 89,737,549 | chr14: 68,683,118 |
| 10 | Cancer | Tier 1 | Tier 1 | Tier 1 | Tier 1 |
| | Gene Tier | | | | |
| 11 | HRR GENE | NO | YES | YES | YES |
| 12 | Linear | 110425 | 283354 | 346647 | N/A break in gene |
| | distance to | | | | |
| | 5′ (bp) | | | | |
| 13 | Closest | 110425 | 283354 | 267549 | N/A break in gene |
| | distance to | | | | |
| | gene body | | | | |
| | (bp) | | | | |
| 14 | Partner 2 | break in N4BP2L2 | break in BRSK2 | break in WIPF3 | break in RPSAP52 |
| | gene or | | | | |
| | intergenic | | | | |
| 15 | Relevant | BRCA2 | N/A | N/A | N/A |
| | cancer | | | | |
| | gene(s) | | | | |
| 16 | Gene 5′ | chr13: 32,315,086 | N/A | N/A | N/A |
| 17 | Gene 3′ | chr13: 32,400,268 | N/A | N/A | N/A |
| 18 | Cancer | Tier 1 | N/A | N/A | N/A |
| | Gene Tier | | | | |
| 19 | HRR GENE | YES | N/A | N/A | N/A |
| 20 | Linear | 154915 | N/A | N/A | N/A Break in |
| | distance to | | | | Gene |
| | 5′ (bp) | | | | |
| 21 | Closest | 69733 | N/A | N/A | N/A Break in |
| | distance to | | | | Gene |
| | gene body | | | | |
| | (bp) | | | | |
| 22 | Approx. | chr13: 32,470,001- | chr11: 1,380,001- | chr7: 29,910,001- | chr12: 65,816,001- |
| | partner | 32,471,00 | 1,390,000 | 29,920,000 | 65,817,000 |
| | breakpoint | | | | |
| | coordinate | | | | |
| | window 2A | | | | |
| 23 | Approx. | chr13: 32,469,001- | chr11: 1,370,001- | chr7: 29,900,001- | chr12: 65,814,001- |
| | partner | 32,472,00 | 1,400,000 | 29,930,000 | 65,819,000 |
| | breakpoint | | | | |
| | coordinate | | | | |
| | window 2B | | | | |
| 24 | genome | Genome-wide | Genome-wide | Genome-wide | Genome-wide |
| | wide or | | | | |
| | capture | | | | |
| 25 | NOTES | N/A | N/A | N/A | N/A |
| 1 | VARIANT | 5 | 6 | 7 | 8 |
| | ID | | | | |
| 2 | SAMPLE | S4 | S5 | S6 | S6 |
| | NUMBER | | | | |
| 3 | Tumor | Undifferentiated | Low-grade | Fibrosarcoma | Fibrosarcoma |
| | type | Uterine Sarcoma | endometrial | | |
| | | (UUS) - Uterine | stromal sarcoma | | |
| | | | (LGESS) - Uterine | | |
| 4 | Partner 1 | break in | intergenic break | break in | break in |
| | type | KREMEN1 | | TRIM37 | RAPGEFL1 |
| 5 | Approx. | chr22: 29,130,001- | chr8: 89,850,001- | chr17: 58,982,001- | chr17: 40,185,001- |
| | breakpoint | 29,135,000 | 89,855,000 | 58,983,000 | 40,190,000 |
| | coordinate | | | | |
| | window 1A | | | | |
| 6 | Approx. | chr22: 29,125,001- | chr8: 89,845,001- | chr17: 58,980,001- | chr17: 40,180,001- |
| | breakpoint | 29,140,000 | 89,860,000 | 58,985,000 | 40,195,000 |
| | coordinate | | | | |
| | window 1B | | | | |
| 7 | Relevant | CHEK2 | NBN | RAD51C | CDK12 |
| | cancer | | | | ERBB2 |
| | gene(s) | | | | |
| 8 | Gene 5′ | chr22: 28,741,820 | chr8: 89,984,682 | chr17: 58,692,602 | CDK12: |
| | | | | | chr17: 39,461,761 |
| | | | | | ERBB2: |
| | | | | | chr17: 39,700,064 |
| 9 | Gene 3′ | chr22: 28,687,743 | chr8: 89,924,515 | chr17: 58,735,611 | CDK12: |
| | | | | | chr17: 39,532,477 |
| | | | | | ERBB2: |
| | | | | | chr17: 39,728,658 |
| 10 | Cancer | Tier 1 | Tier 1 | Tier 1 | CDK12: Tier 1 |
| | Gene Tier | | | | ERBB2: Tier 1 |
| 11 | HRR | YES | YES | YES | CDK12: YES |
| | GENE | | | | ERBB2: NO |
| 12 | Linear | 388181 | 129682 | 289399 | CDK12: 723,240 |
| | distance | | | | ERBB2: 484,937 |
| | to 5′ (bp) | | | | |
| 13 | Closest | 388181 | 69515 | 246390 | CDK12: 652,524 |
| | distance | | | | ERBB2: 456,343 |
| | to gene | | | | |
| | body (bp) | | | | |
| 14 | Partner 2 | intergenic break | break in | break in | intergenic break |
| | gene or | | TSNARE1 | PITPNC1 | |
| | intergenic | | | | |
| 15 | Relevant | SMARCA4 | N/A | N/A | SUZ12 |
| | cancer | | | | |
| | gene(s) | | | | |
| 16 | Gene 5′ | chr19: 10,961,001 | N/A | N/A | chr17: 31,937,007 |
| 17 | Gene 3′ | chr19: 11,062,256 | N/A | N/A | chr17: 32,001,038 |
| 18 | Cancer | Tier 3 | N/A | N/A | Tier 3 |
| | Gene Tier | | | | |
| 19 | HRR | NO | N/A | N/A | NO |
| | GENE | | | | |
| 20 | Linear | 569000 | N/A Break in | N/A Break in | 112007 |
| | distance | | Gene | Gene | |
| | to 5′ (bp) | | | | |
| 21 | Closest | 467745 | N/A Break in | N/A Break in | 112007 |
| | distance | | Gene | Gene | |
| | to gene | | | | |
| | body (bp) | | | | |
| 22 | Approx. | chr19: 11,530,001- | chr8: 142,210,001- | chr17: 67,659,001- | chr17: 31,820,001- |
| | partner | 11,535,000 | 142,215,008 | 67,660,000 | 31,825,000 |
| | breakpoint | | | | |
| | coordinate | | | | |
| | window 2A | | | | |
| 23 | Approx. | chr19: 11,525,001- | chr8: 142,205,001- | chr17: 67,657,001- | chr17: 31,815,001- |
| | partner | 11,540,000 | 142,220,008 | 67,662,000 | 31,830,000 |
| | breakpoint | | | | |
| | coordinate | | | | |
| | window 2B | | | | |
| 24 | genome | Genome-wide | Genome-wide | Genome-wide | Genome-wide |
| | wide or | | | | |
| | capture | | | | |
| 25 | NOTES | N/A | N/A | N/A | N/A |

TABLE 8.2

| Row | | | | | |
|---|---|---|---|---|---|
| 1 | VARIANT | 9 | 10 | 11 | 12 |
| | ID | | | | |
| 2 | SAMPLE | S7 | S8 | S9 | S10 |
| | NUMBER | | | | |
| 3 | Tumor | Sarcoma with | Low Grade | Highly atypical | High grade |
| | type | sex-cord like | Adenosarcoma | spindled and | Adenosarcoma |
| | | differentiation | | epithelioid | with sarcoma |
| | | | | neoplasm with | overgrowth (HG |
| | | | | myxoid features, | ASSO) |
| | | | | c/w sarcoma | |
| 4 | Partner 1 | break in CCT6B | intergenic break | break in RAD51B | intergenic break |
| | type | | | | |
| 5 | Approx. | chr17: 34,940,001- | chr14: 68,753,001- | chr14: 68,324,001- | chr8: 56,137,001- |
| | breakpoint | 34,945,000 | 68,754,000 | 68,325,000 | 56,138,000 |
| | coordinate | | | | |
| | window 1A | | | | |
| 6 | Approx. | chr17: 34,935,001- | chr14: 68,751,001- | chr14: 68,322,001- | chr8: 56, 135,001- |
| | breakpoint | 34,950,000 | 68,756,000 | 68,327,000 | 56,140,000 |
| | coordinate | | | | |
| | window 1B | | | | |
| 7 | Relevant | RAD51D | RAD51B | RAD51B | PLAG1 |
| | cancer | | | | |
| | gene(s) | | | | |
| 8 | Gene 5′ | chr17: 35,119,860 | chr14: 67,865,032 | chr14: 67,865,032 | chr8: 56,211,273 |
| 9 | Gene 3′ | chr17: 35,092,221 | chr14: 68,683,118 | chr14: 68,683,118 | chr8: 56,160,909 |
| 10 | Cancer | Tier 1 | Tier 1 | Tier 1 | Tier 3 |
| | Gene Tier | | | | |
| 11 | HRR | YES | YES | YES | NO |
| | GENE | | | | |
| 12 | Linear | 174860 | 887969 | N/A break in gene | 73273 |
| | distance | | | | |
| | to 5′ (bp) | | | | |
| 13 | Closest | 147221 | 69883 | N/A break in gene | 22909 |
| | distance | | | | |
| | to gene | | | | |
| | body (bp) | | | | |
| 14 | Partner 2 | break in | break in | break in | break in |
| | gene or | PIMREG | RPSAP52 | NRXN3 | RAD51B |
| | intergenic | | | | |
| 15 | Relevant | N/A | N/A | N/A | RAD51B |
| | cancer | | | | |
| | gene(s) | | | | |
| 16 | Gene 5′ | N/A | N/A | N/A | chr14: 67,865,032 |
| 17 | Gene 3′ | N/A | N/A | N/A | chr14: 68,683,118 |
| 18 | Cancer | N/A | N/A | N/A | Tier 1 |
| | Gene Tier | | | | |
| 19 | HRR | N/A | N/A | N/A | YES |
| | GENE | | | | |
| 20 | Linear | N/A Break in | N/A Break in | N/A Break in | N/A Break in |
| | distance | Gene | Gene | Gene | Gene |
| | to 5′ (bp) | | | | |
| 21 | Closest | N/A Break in | N/A Break in | N/A Break in | N/A Break in |
| | distance | Gene | Gene | Gene | Gene |
| | to gene | | | | |
| | body (bp) | | | | |
| 22 | Approx. | chr17: 6,445,001- | chr12: 65,811,001- | chr14: 79,637,001- | chr14: 68,478,001- |
| | partner | 6,450,000 | 65,812,000 | 79,638,000 | 68,479,000 |
| | breakpoint | | | | |
| | coordinate | | | | |
| | window 2A | | | | |
| 23 | Approx. | chr17: 6,440,001- | chr12: 65,809,001- | chr14: 79,635,001- | chr14: 68,476,001- |
| | partner | 6,455,000 | 65,814,000 | 79,640,000 | 68,481,000 |
| | breakpoint | | | | |
| | coordinate | | | | |
| | window 2B | | | | |
| 24 | genome | Genome-wide | Genome-wide | Genome-wide | Genome-wide |
| | wide or | | | | |
| | capture | | | | |
| 25 | NOTES | N/A | N/A | N/A | N/A |

TABLE 8.3

| Row | | | | | |
|---|---|---|---|---|---|
| 1 | VARIANT | 13 | 14 | 15 | 17 |
| | ID | | | | |
| 2 | SAMPLE | S10 | S11 | S11 | S12 |
| | NUMBER | | | | |
| 3 | Tumor | High grade | Adenosarcoma | Adenosarcoma | Uterine smooth |
| | type | Adenosarcoma | with sarcoma | with sarcoma | muscle tumor of |
| | | with sarcoma | overgrowth | overgrowth (ASSO) | uncertain |
| | | overgrowth (HG | (ASSO) | | malignant |
| | | ASSO) | | | potential |
| | | | | | (STUMP) |
| 4 | Partner 1 | break in | intergenic break | break in RAB39A | break in FANCA |
| | type | GLYCTK-AS1 | | | |
| 5 | Approx. | chr3: 52,289,001- | chr17: 61,610,001- | chr11: 107,955,001- | chr16: 89,791,001- |
| | breakpoint | 52,290,000 | 61,615,000 | 107,960,000 | 89,792,000 |
| | coordinate | | | | |
| | window 1A | | | | |
| 6 | Approx. | chr3: 52,287,001- | chr17: 61,605,001- | chr11: 107,950,001- | chr16: 89,789,001- |
| | breakpoint | 52,292,000 | 61,620,000 | 107,965,000 | 89,794,000 |
| | coordinate | | | | |
| | window 1B | | | | |
| 7 | Relevant | PARP3 | BRIP1 | ATM | FANCA |
| | cancer | | | | |
| | gene(s) | | | | |
| 8 | Gene 5′ | chr3: 51,942,345 | chr17: 61,863,528 | chr11: 108,223,067 | chr16: 89,816,647 |
| 9 | Gene 3′ | chr3: 51,948,862 | chr17: 61,679,139 | chr11: 108,369,102 | chr16: 89,737,549 |
| 10 | Cancer | Tier 1 | Tier 1 | Tier 1 | Tier 1 |
| | Gene Tier | | | | |
| 11 | HRR | YES | YES | YES | YES |
| | GENE | | | | |
| 12 | Linear | 346656 | 248528 | 263067 | N/A Break in |
| | distance | | | | Gene |
| | to 5′ (bp) | | | | |
| 13 | Closest | 340139 | 64139 | 263067 | N/A Break in |
| | distance | | | | Gene |
| | to gene | | | | |
| | body (bp) | | | | |
| 14 | Partner 2 | intergenic break | break in VGLL4 | intergenic break | intergenic break |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer | N/A | N/A | N/A | N/A |
| | Gene Tier | | | | |
| 19 | HRR | N/A | N/A | N/A | N/A |
| | GENE | | | | |
| 20 | Linear | NA | N/A Break in | N/A | N/A |
| | distance | | Gene | | |
| | to 5′ (bp) | | | | |
| 21 | Closest | N/A | N/A Break in | N/A | N/A |
| | distance | | Gene | | |
| | to gene | | | | |
| | body (bp) | | | | |
| 22 | Approx. | chr3: 42,036,001- | chr3: 11,625,001- | chr11: 110,975,001- | chr13: 44,810,001- |
| | partner | 42,037,000 | 11,630,000 | 110,980,000 | 44,811,000 |
| | breakpoint | | | | |
| | coordinate | | | | |
| | window 2A | | | | |
| 23 | Approx. | chr3: 42,034,001- | chr3: 11,620,001- | chr11: 110,970,001- | chr13: 44,808,001- |
| | partner | 42,039,000 | 11,635,000 | 110,985,000 | 44,813,000 |
| | breakpoint | | | | |
| | coordinate | | | | |
| | window 2B | | | | |
| 24 | genome | Genome-wide | Genome-wide | Genome-wide | Genome-wide |
| | wide or | | | | |
| | capture | | | | |
| 25 | NOTES | N/A | N/A | N/A | N/A |

TABLE 8.4

| Row | | | | | |
|---|---|---|---|---|---|
| 1 | VARIANT | 18 | 19 | 20 | 21 |
| | ID | | | | |
| 2 | SAMPLE | S13 | S14 | S15 | S16 |
| | NUMBER | | | | |
| 3 | Tumor | Uterine smooth | High-grade | Atypical | High grade |
| | type | muscle tumor of | endometrial | leiomyosarcoma | adenosarcoma |
| | | uncertain | stromal sarcoma | (LM) with low | (HG AS) |
| | | malignant | (HGESS) - | recurrence risk | |
| | | potential | Uterine | | |
| | | (STUMP) | | | |
| 4 | Partner 1 | break in RAD51B | intergenic break | break in RAD51B | break in RAD51B |
| | type | | | | |
| 5 | Approx. | chr14: 68,650,001- | chr3: 10,140,001- | chr14: 68,660,001- | chr14: 68,390,001- |
| | breakpoint | 68,655,000 | 10,145,000 | 68,665,000 | 68,391,000 |
| | coordinate | | | | |
| | window 1A | | | | |
| 6 | Approx. | chr14: 68,645,001- | chr3: 10,135,001- | chr14: 68,655,001- | chr14: 68,388,001- |
| | breakpoint | 68,660,000 | 10,150,000 | 68,670,000 | 68,393,000 |
| | coordinate | | | | |
| | window 1B | | | | |
| 7 | Relevant | RAD51B | FANCD2 | RAD51B | RAD51B |
| | cancer | | | | |
| | gene(s) | | | | |
| 8 | Gene 5′ | chr14: 67,865,032 | chr3: 10,026,437 | chr14: 67,865,032 | chr14: 67,865,032 |
| 9 | Gene 3′ | chr14: 68,683,118 | chr3: 10,101,932 | chr14: 68,683,118 | chr14: 68,683,118 |
| 10 | Cancer | Tier 1 | Tler 1 | Tier 1 | Tler 1 |
| | Gene Tier | | | | |
| 11 | HRR | YES | YES | YES | YES |
| | GENE | | | | |
| 12 | Linear | N/A Break in | 113564 | N/A Break in | N/A Break in |
| | distance | Gene | | Gene | Gene |
| | to 5′ (bp) | | | | |
| 13 | Closest | N/A Break in | 38069 | N/A Break in | N/A Break in |
| | distance | Gene | | Gene | Gene |
| | to gene | | | | |
| | body (bp) | | | | |
| 14 | Partner 2 | intergenic break | break in ADCY1 | break in NUDT3 | break in ME3 |
| | gene or | | | | |
| | intergenic | | | | |
| 15 | Relevant | N/A | N/A | N/A | N/A |
| | cancer | | | | |
| | gene(s) | | | | |
| 16 | Gene 5′ | N/A | N/A | N/A | N/A |
| 17 | Gene 3′ | N/A | N/A | N/A | N/A |
| 18 | Cancer | N/A | N/A | N/A | N/A |
| | Gene Tier | | | | |
| 19 | HRR | N/A | N/A | N/A | N/A |
| | GENE | | | | |
| 20 | Linear | N/A | N/A Break in | N/A Break in | N/A Break in |
| | distance | | Gene | Gene | Gene |
| | to 5′ (bp) | | | | |
| 21 | Closest | N/A | N/A Break in | N/A Break in | N/A Break in |
| | distance | | Gene | Gene | Gene |
| | to gene | | | | |
| | body (bp) | | | | |
| 22 | Approx. | chr12: 65,725,001- | chr7: 45,680,001- | chr6: 34,365,001- | chr11: 86,463,001- |
| | partner | 65,730,000 | 45,685,000 | 34,370,000 | 86,464,000 |
| | breakpoint | | | | |
| | coordinate | | | | |
| | window 2A | | | | |
| 23 | Approx. | chr12: 65,720,001- | chr7: 45,675,001- | chr6: 34,360,001- | chr11: 86,461,001- |
| | partner | 65,735,000 | 45,690,000 | 34,375,000 | 86,466,000 |
| | breakpoint | | | | |
| | coordinate | | | | |
| | window 2B | | | | |
| 24 | genome | Genome-wide | Genome-wide | Genome-wide | Genome-wide |
| | wide or | | | | |
| | capture | | | | |
| 25 | NOTES | N/A | N/A | N/A | N/A |

TABLE 8.5

| Row | | | | | |
|---|---|---|---|---|---|
| 1 | VARIANT ID | 22 | 23 | 24 | 25 |
| 2 | SAMPLE | S17 | S18 | S19 | S19 |
| | NUMBER | | | | |
| 3 | Tumor type | Undifferentiated | Adenosarcoma | Myxoid | Myxoid |
| | | Uterine Sarcoma | with sarcoma | leiomyosarcoma | leiomyosarcoma |
| | | (UUS) - Uterine | overgrowth | (LMS) | (LMS) |
| | | | (ASSO) | | |
| 4 | Partner 1 | break in CDON | break in ATM | Intergenic break | break in RP1 |
| | type | | | | |
| 5 | Approx. | chr11: 126,000,001- | chr11 :108,275,001- | chr8: 56,129,001- | chr8: 54,695,001- |
| | breakpoint | 126,005,000 | 108,280,000 | 56,130,000 | 54,700,000 |
| | coordinate | | | | |
| | window 1A | | | | |
| 6 | Approx. | chr11: 125,995,001- | chr11: 108,270,001- | chr8: 56,127,001- | chr8: 54,690,001- |
| | breakpoint | 126,010,000 | 108,285,000 | 56,132,000 | 54,705,000 |
| | coordinate | | | | |
| | window 1B | | | | |
| 7 | Relevant | CHEK1 | ATM | PLAG1 | N/A |
| | cancer | | | | |
| | gene(s) | | | | |
| 8 | Gene 5′ | chr11: 125,625,974 | chr11: 108,223,067 | PLAG1: | N/A |
| | | | | chr8: 56,211,273 | |
| 9 | Gene 3′ | chr11: 125,676,255 | chr11: 108,369,102 | PLAG1: | N/A |
| | | | | chr8: 56,160,909 | |
| 10 | Cancer Gene | Tier 1 | Tier 1 | Tier 3 | N/A |
| | Tier | | | | |
| 11 | HRR GENE | YES | YES | NO | N/A |
| 12 | Linear | 374027 | N/A Break In Gene | 81273 | N/A |
| | distance to 5′ | | | | |
| | (bp) | | | | |
| 13 | Closest | 323746 | N/A Break in Gene | 30909 | N/A |
| | distance to | | | | |
| | gene body | | | | |
| | (bp) | | | | |
| 14 | Partner 2 | break in | break in | break in | break in |
| | gene or | GAB2 | MSANTD2 | RAD51B | RAD51B |
| | intergenic | | | | |
| 15 | Relevant | N/A | N/A | RAD51B | RAD51B |
| | cancer | | | | |
| | gene(s) | | | | |
| 16 | Gene 5′ | N/A | N/A | chr14: 67,865,032 | chr14: 67,865,032 |
| 17 | Gene 3′ | N/A | N/A | chr14: 68,683,118 | chr14: 68,683,118 |
| 18 | Cancer Gene | N/A | N/A | Tier 1 | Tier 1 |
| | Tier | | | | |
| 19 | HRR GENE | N/A | N/A | YES | YES |
| 20 | Linear | N/A Break in | N/A Break in | N/A Break in | N/A Break in |
| | distance to 5′ | Gene | Gene | Gene | Gene |
| | (bp) | | | | |
| 21 | Closest | N/A Break in | N/A Break in | N/A Break in | N/A Break in |
| | distance to | Gene | Gene | Gene | Gene |
| | gene body | | | | |
| | (bp) | | | | |
| 22 | Approx. | chr11: 78,415,001- | chr11: 124,785,001- | chr14: 68,523,001- | chr14: 68,525,001- |
| | partner | 78,420,000 | 124,790,000 | 68,524,000 | 68,530,000 |
| | breakpoint | | | | |
| | coordinate | | | | |
| | window 2A | | | | |
| 23 | Approx. | chr11: 78,410,001- | chr11: 124,780,001- | chr14: 68,521,001- | chr14: 68,520,001- |
| | partner | 78,425,000 | 124,795,000 | 68,526,000 | 68,535,000 |
| | breakpoint | | | | |
| | coordinate | | | | |
| | window 2B | | | | |
| 24 | genome wide | Genome-wide | Genome-wide | Genome-wide | Genome-wide |
| | or capture | | | | |
| 25 | NOTES | N/A | N/A | 10 | N/A |

TABLE 8.6

| Row | | | | | |
|---|---|---|---|---|---|
| 1 | VARIANT | 26 | 27 | 28 | 29 |
| | ID | | | | |
| 2 | SAMPLE | S20 | S21 | S22 | S23 |
| | NUMBER | | | | |
| 3 | Tumor | leiomyosarcoma | Diffuse large B | Chordoma | Uterine Myxoid |
| | type | (LMS) | cell lymphoma | | Leiomyosarcoma |
| 4 | Partner 1 | break in | break in | break in | break in |
| | type | TATDN2 | TSPOAP1-AS1 | LEXM | LYN |
| 5 | Approx. | chr3: 10,267,001- | chr17: 58,332,001- | chr1: 54,805,001- | chr8: 55,988,001- |
| | breakpoint | 10,268,000 | 58,333,000 | 54,810,000 | 55,991,000 |
| | coordinate | | | | |
| | window 1A | | | | |
| 6 | Approx. | chr3: 10,264,001- | chr17: 58,329,001- | chr1: 54,800,001- | chr8: 55,985,001- |
| | breakpoint | 10,271,000 | 58,337,000 | 54,815,000 | 55,994,000 |
| | coordinate | | | | |
| | window 1B | | | | |
| 7 | Relevant | VHL | RAD51C | N/A | LYN |
| | cancer | FANCD2 | RNF43 | | PLAG1 |
| | gene(s) | | | | |
| 8 | Gene 5′ | VHL: | RAD51C: | N/A | LYN: |
| | | chr3: 10,141,778 | chr17: 58,692,602 | | chr8: 55,879,835 |
| | | FANCD2: | RNF43: | | PLAG1: |
| | | chr3: 10,026,437 | chr17: 58,417,582 | | chr8: 56,211,273 |
| 9 | Gene 3′ | VHL: | RAD51C: | N/A | LYN: |
| | | chr3: 10,153,667 | chr17: 58,735,611 | | chr8: 55,879,835 |
| | | FANCD2: | RNF43: | | PLAG1: |
| | | chr3: 10,101,932 | chr17: 58,353,676 | | chr8: 56,160,909 |
| 10 | Cancer | VHL: Tier 1 | RAD51C: Tier 1 | N/A | LYN: Tier 4 |
| | Gene Tier | FANCD2: Tier 1 | RNF43: Tier 4 | | PLAG1: Tier 3 |
| 11 | HRR | VHL: No | RAD51C: YES | N/A | NO |
| | GENE | FANCD2: Yes | RNF43: NO | | |
| 12 | Linear | VHL: 125,223 | RAD51C: 359,602 | N/A | LYN: N/A Break |
| | distance | FANCD2: 240,564 | RNF43: 84,582 | | in Gene |
| | to 5′ (bp) | | | | PLAG1: 220,273 |
| 13 | Closest | VHL: 113,334 | RAD51C: 359,602 | N/A | LYN: N/A Break in |
| | distance | FANCD2: 165,069 | RNF43: 20,676 | | Gene |
| | to gene | | | | PLAG1: 169,909 |
| | body (bp) | | | | |
| 14 | Partner 2 | intergenic break | break in | break in | break in |
| | gene or | | COPE | RAD54L | RAD51B |
| | intergenic | | | | |
| 15 | Relevant | N/A | MEF2B | RAD54L | RAD51B |
| | cancer | | | MKNK1 | |
| | gene(s) | | | | |
| 16 | Gene 5′ | N/A | chr19: 19,192,131 | RAD54L: | chr14: 67,865,032 |
| | | | | chr1: 46,247,700 | |
| | | | | MKNK1: | |
| | | | | chr1: 46,604,268 | |
| 17 | Gene 3′ | N/A | chr19: 19,145,567 | RAD54L: | chr14: 68,683,118 |
| | | | | chr1: 46,278,480 | |
| | | | | MKNK1: | |
| | | | | chr1: 46,557,407 | |
| 18 | Cancer | N/A | Tier 4 | RAD54L: Tier 1 | Tier 1 |
| | Gene Tier | | | MKNK1: Tier 4 | |
| 19 | HRR | N/A | NO | RAD54L: YES | YES |
| | GENE | | | MKNK1: NO | |
| 20 | Linear | N/A | 288131 | RAD54L: N/A | N/A Break in |
| | distance | | | break in gene | Gene |
| | to 5′ (bp) | | | MKNK1: 344,268 | |
| 21 | Closest | N/A | 241567 | RAD54L: N/A | N/A Break in |
| | distance | | | break in gene | Gene |
| | to gene | | | MKNK1: 297,407 | |
| | body (bp) | | | | |
| 22 | Approx. | chr8: 129,181,001- | chr19: 18,903,001- | chr1: 46,255,001- | chr14: 68,632,001- |
| | partner | 129,182,000 | 18,904,000 | 46,260,000 | chr14: 68,635,000 |
| | breakpoint | | | | |
| | coordinate | | | | |
| | window 2A | | | | |
| 23 | Approx. | chr8: 129,178,001- | chr19: 18,900,001- | chr1: 46,250,001- | chr14: 68,629,001- |
| | partner | 129,185,000 | 18,907,000 | 46,265,000 | chr14:68,638,000 |
| | breakpoint | | | | |
| | coordinate | | | | |
| | window 2B | | | | |
| 24 | genome | Genome-wide | Genome-wide | Genome-wide | Genome-wide |
| | wide or | | | | |
| | capture | | | | |
| 25 | NOTES | N/A | N/A | N/A | 14, 34 |

TABLE 8.7

| Row | | | | | |
|---|---|---|---|---|---|
| 1 | VARIANT | 30 | 31 | 32 | 33 |
| | ID | | | | |
| 2 | SAMPLE | S24 | S24 | S25 | S26 |
| | NUMBER | | | | |
| 3 | Tumor | Uterine Myxoid | Uterine Myxoid | Uterine Myxoid | Chordoma |
| | type | Leiomyosarcoma | Leiomyosarcoma | Leiomyosarcoma | |
| 4 | Partner 1 | Intergenic Break | Intergenic Break | break in PDS5A | Intergenic Break |
| | type | | | | |
| 5 | Approx. | chr12: 65,756,001- | chr12: 48,190,001- | chr4: 39,974,001- | chr3: 51,788,001- |
| | breakpoint | 65,757,000 | 48,195,000 | 39,975,000 | 51,790,000 |
| | coordinate | | | | |
| | window 1A | | | | |
| 6 | Approx. | chr12: 65,754,001- | chr12: 48,190,001- | chr4: 39,972,001- | chr3: 51,786,001- |
| | breakpoint | 65,759,000 | 48,195,000 or | 39,977,000 | 51,792,000 |
| | coordinate | | chr12: 48,185,001- | | |
| | window 1B | | 48,200,000 | | |
| 7 | Relevant | N/A | N/A | N/A | PARP3 |
| | cancer | | | | |
| | gene(s) | | | | |
| 8 | Gene 5′ | N/A | N/A | N/A | chr3: 51,942,345 |
| 9 | Gene 3′ | N/A | N/A | N/A | chr3: 51,948,862 |
| 10 | Cancer | N/A | N/A | N/A | Tier 1 |
| | Gene Tier | | | | |
| 11 | HRR | N/A | N/A | N/A | YES |
| | GENE | | | | |
| 12 | Linear | N/A | N/A | N/A | 152345 |
| | distance | | | | |
| | to 5′ (bp) | | | | |
| 13 | Closest | N/A | N/A | N/A | 152345 |
| | distance | | | | |
| | to gene | | | | |
| | body (bp) | | | | |
| 14 | Partner 2 | break in RAD51B | break in RAD51B | Intergenic | Intergenic |
| | gene or | | | | |
| | intergenic | | | | |
| 15 | Relevant | RAD51B | RAD51B | RAD51D | CRBN |
| | cancer | | | | |
| | gene(s) | | | | |
| 16 | Gene 5′ | chr14: 67,865,032 | chr14: 67,865,032 | chr17: 35,119,860 | chr3: 3,179,691 |
| 17 | Gene 3′ | chr14: 68,683,118 | chr14: 68,683,118 | chr17: 35,092,221 | chr3: 3,150,011 |
| 18 | Cancer | Tier 1 | Tier 1 | Tier 1 | Tier 4 |
| | Gene Tier | | | | |
| 19 | HRR | YES | YES | YES | NO |
| | GENE | | | | |
| 20 | Linear | N/A (break in | N/A (break in | 25141 | 91310 |
| | distance | gene) | gene) | | |
| | to 5′ (bp) | | | | |
| 21 | Closest | N/A (break in | N/A (break in | 25141 | 91310 |
| | distance | gene) | gene) | | |
| | to gene | | | | |
| | body (bp) | | | | |
| 22 | Approx. | chr14: 68,673,001- | chr14: 68,675,001- | chr17: 35,145,001- | chr3: 3,271,001- |
| | partner | 68,674,000 | 68,680,000 | 35,146,000 | 3,273,000 |
| | breakpoint | | | | |
| | coordinate | | | | |
| | window 2A | | | | |
| 23 | Approx. | chr14: 68,671,001- | chr14: 68,670,001- | chr17: 35,143,001- | chr3: 3,269,001- |
| | partner | 68,676,000 | 68,685,000 | 35,148,000 | 3,275,000 |
| | breakpoint | | | | |
| | coordinate | | | | |
| | window 2B | | | | |
| 24 | genome | Genome-wide | Genome-wide | Genome-wide | Genome-wide |
| | wide or | | | | |
| | capture | | | | |
| 25 | NOTES | 22, 34 | 23, 34 | 34 | 28 |

TABLE 8.8

| Row | | | |
|---|---|---|---|
| 1 | VARIANT ID | 34 | 35 |
| 2 | SAMPLE | S27 | S28 |
| | NUMBER | | |
| 3 | Tumor type | Chrodoma | Meningioma |
| 4 | Partner 1 | Break in MAST2 | break in ITGA3 |
| | type | | |
| 5 | Approx. | chr1: 45,920,001- | chr17: 50,058,001- |
| | breakpoint | 45,930,000 | 50,061,000 |
| | coordinate | | |
| | window 1A | | |
| 6 | Approx. | chr1: 45,910,001- | chr17: 50,056,001- |
| | breakpoint | 45,940,000 | 50,063,000 |
| | coordinate | | |
| | window 1B | | |
| 7 | Relevant | RAD54L | N/A |
| | cancer | | |
| | gene(s) | | |
| 8 | Gene 5′ | chr1: 46,247,700 | N/A |
| 9 | Gene 3′ | chr1: 46,278,480 | N/A |
| 10 | Cancer Gene | Tier 1 | N/A |
| | Tier | | |
| 11 | HRR GENE | YES | N/A |
| 12 | Linear | 317,700 | N/A |
| | distance to 5′ | | |
| | (bp) | | |
| 13 | Closest | 317,700 | N/A |
| | distance to | | |
| | gene body | | |
| | (bp) | | |
| 14 | Partner 2 | Intergenic | Intergenic |
| | gene or | | |
| | intergenic | | |
| 15 | Relevant | N/A | CDK12 |
| | cancer | | |
| | gene(s) | | |
| 16 | Gene 5′ | N/A | chr17: 39,461,486 |
| 17 | Gene 3′ | N/A | chr17: 39,534,544 |
| 18 | Cancer Gene | N/A | Tier 4 |
| | Tier | | |
| 19 | HRR GENE | N/A | YES |
| 20 | Linear | N/A | 130515 |
| | distance to 5′ | | |
| | (bp) | | |
| 21 | Closest | N/A | 57457 |
| | distance to | | |
| | gene body | | |
| | (bp) | | |
| 22 | Approx. | chr1: 164,320,001- | chr17: 39,592,001- |
| | partner | 164,330,000 | 39,593,000 |
| | breakpoint | | |
| | coordinate | | |
| | window 2A | | |
| 23 | Approx. | chr1: 164,310,001- | chr17: 39,590,001- |
| | partner | 164,340,000 | 39,595,000 |
| | breakpoint | | |
| | coordinate | | |
| | window 2B | | |
| 24 | genome wide | Genome-wide | Genome-wide |
| | or capture | | |
| 25 | NOTES | N/A | 29 |

Notes from Table X:

10. Translocation forms RP1-RAD51B gene fusion.

14. Translocation, resulting in an in-frame gene fusion with RAD51B as the 5′ partner and LYN as the 3′ partner. Lyn is a tyrosine kinase and a known 3′ fusion partner in hematologic cancers. The tyrosine kinas domain is in the 3′ portion of LYN. Not aware of any reports of Lyn fusions in sarcomas. LYN is also involved in a complex rearrangement involving ZFPM2 on chr8 and ARFGEF1 also on chr8.

22. This translocation has a breakpoint in RAD51B, and the 5′ portion of RAD51B is involved in the rearrangement.

23. This translocation has a breakpoint in RAD51B, and the 3′ portion of RAD51B is involved in the rearrangement. This could be a complex rearrangement with variant 30.

28. This SV is an inversion.

29. This structural variant is a deletion—the segment between the breakpoints has been deleted.

34. Notable trends in the 4 uterine myxoid LMS tumors: RAD51 alterations were found in 3/4 tumors, with 2 involving RAD51B and 1 with RAD51D. Two with breakpoints within RAD51 genes, and one with breakpoint adjacent to the gene. PRKD gene fusions observed in 2/4 samples. One was PRKD1 and the other PRKDC. Highly rearranged chr8 (with numerous intra- and inter-chromosomal rearrangements) in 2/4 samples (S23 and S24).

The entirety of each patent, patent application, publication and document referenced herein is incorporated by reference, to the extent permitted by law. Citations of patents, patent applications, publications and documents are not an admission that any of the foregoing is pertinent prior art, nor does it constitute any admission as to the contents or date of these publications or documents. Their citation is not an indication of a search for relevant disclosures. All statements regarding the date(s) or contents of the documents are based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein. Unless otherwise noted, technical terms are used according to conventional usage. Definitions of common terms in molecular biology may be found in Benjamin Lewin's Genes XII, published by Jones & Bartlett Learning, 2017 (ISBN-10: 1284104494) and Joseph Jez (ed), Encyclopedia of Biological Chemistry, published by Elsevier, 2021 (ISBN 9780128194607).

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80/a, 85% or 90%” includes the intermediate value 86% and the fractional value 86.4%). When a listing of values is followed by the term “or more.” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

1-20. (canceled)

21. A method of treating a subject that has, or is suspected of having, cancer, the method comprising: a) identifying and/or selecting a subject comprising a genome with a fusion, wherein the fusion comprises at least a portion of a homologous recombinant repair gene; and b) treating the subject so identified and/or selected with a homologous recombination deficient directed therapy.

22. The method of claim 21, wherein identifying and/or selecting a subject comprising a genome with a fusion comprises: i) performing a nucleic acid analysis on a sample obtained from the subject; and ii) detecting whether the fusion is present or absent in the sample according to the analysis in i).

23. The method of claim 22, wherein the nucleic acid analysis is selected from the group consisting of: nucleic acid sequencing, optical genome mapping, amplification, microarray analysis, and fluorescence in situ hybridization.

24. The method of claim 23, wherein the nucleic acid sequencing is targeted sequencing or genome-wide sequencing.

25. The method of claim 22, wherein the nucleic acid analysis in a) comprises a method that preserves spatial-proximal contiguity information.

26. The method of claim 25, wherein the method that preserves spatial-proximal contiguity information is a proximity ligation method.

27. The method of claim 26, wherein the method that preserves spatial-proximal contiguity information is Hi-C or 3C.

28. The method of claim 21, wherein the fusion comprising at least a portion of a homologous recombinant repair gene is to a gene or portion thereof.

29. The method of claim 28, wherein the fusion compromises the function of the homologous recombination gene.

30. The method of claim 28, wherein the fusion is not in frame.

31. The method of claim 21, wherein the fusion comprising at least a portion of a homologous recombinant repair gene is to an intergenic region.

32. The method of claim 21, wherein the homologous recombinant repair gene is selected from the group consisting of: ATM, BRCA2, CHEK2, NBN, RAD51C, SEM1, ATR, BRIP1, FANCA, PALB2, RAD51D, BARD1, CDK12, FANCD2, PARP3, RAD54L, BRCA1, CHEK1, FANCL, RAD51B, and RPA1.

33. The method of claim 32, wherein the homologous recombinant repair gene is RAD51B.

34. The method of claim 21, wherein the cancer is selected from the group consisting of: Acute Lymphoblastic Leukemia; Acute Myeloid Leukemia; Ampullary Adenocarcinoma; Anal Carcinoma; Basal Cell Skin Cancer; B-Cell Lymphomas; Biliary Tract Cancers; Bladder Cancer; Bone Cancer; Breast Cancer: Castleman Disease; Central Nervous System Cancers; Cervical Cancer; Chronic Lymphocytic Leukemia/Small Lymphocytic Lymphoma; Chronic Myeloid Leukemia; Colon Cancer; Dermatofibrosarcoma Protuberans; Esophageal and Esophagogastric Junction Cancers; Gastric Cancer; Gastrointestinal Stromal Tumors; Gestational Trophoblastic Neoplasia; Hairy Cell Leukemia; Head and Neck Cancers; Hepatobiliary Cancers; Hepatocellular Carcinoma; Histiocytic Neoplasms; Hodgkin Lymphoma; Kaposi Sarcoma; Kidney Cancer; Melanoma: Cutaneous; Melanoma: Uveal; Merkel Cell Carcinoma; Mesothelioma: Peritoneal; Mesothelioma: Pleural; Multiple Myeloma; Myelodysplastic Syndromes; Myeloid/Lymphoid Neoplasms with Eosinophilia and Tyrosine Kinase Gene Fusions; Myeloproliferative Neoplasms; Neuroblastoma; Neuroendocrine and Adrenal Tumors; Non-Small Cell Lung Cancer; Occult Primary; Ovarian Cancer/Fallopian Tube Cancer/Primary Peritoneal Cancer; Pancreatic Adenocarcinoma; Pediatric Acute Lymphoblastic Leukemia; Pediatric Aggressive Mature B-Cell Lymphomas; Pediatric Central Nervous System Cancers; Pediatric Hodgkin Lymphoma; Penile Cancer; Primary Cutaneous Lymphomas; Prostate Cancer; Rectal Cancer; Small Bowel Adenocarcinoma; Small Cell Lung Cancer; Soft Tissue Sarcoma; Squamous Cell Skin Cancer; Systemic Light Chain Amyloidosis; Systemic Mastocytosis; T-Cell Lymphomas; Testicular Cancer; Thymomas and Thymic Carcinomas; Thyroid Carcinoma; Uterine Neoplasms; Vaginal Cancer; Vulvar Cancer; Waldenström Macroglobulinemia/Lymphoplasmacytic Lymphoma; or Wilms Tumor (Nephroblastoma).

35. The method of claim 21, wherein the cancer is selected from the group consisting of: kidney cancer, a cancer of the central nervous system (CNS), breast cancer, colorectal cancer, gastric cancer, lung cancer, thyroid cancer, and testicular cancer.

36. The method of claim 21, wherein the cancer is selected from the group consisting of: colorectal carcinoma, chordoma, undifferentiated uterine sarcoma, low-grade endometrial stromal sarcoma, fibrosarcoma, sarcoma with sex-cord like differentiation, low grade adenosarcoma, highly atypical spindled and epithelioid neoplasm with myxoid features; high grade adenosarcoma with sarcoma overgrowth, adenosarcoma with sarcoma overgrowth, uterine smooth muscle tumor, high-grade endometrial stromal sarcoma, atypical leiomyosarcoma, high grade adenosarcoma, myxoid leiomyosarcoma, leiomyosarcoma, diffuse large B cell lymphoma, uterine myxoid leiomyosarcoma, and meningioma.

37. The method of claim 21, wherein the homologous recombination deficient directed therapy is a therapy that is efficacious in the treatment of cancer.

38. The method of claim 37, wherein the homologous recombination deficient directed therapy is a therapy selected from the group consisting of: an inhibitor of a polyadenosine diphosphate-ribose polymerase (PARP) enzyme and a platinum-based therapy.

39. The method of claim 38, wherein the inhibitor of a polyadenosine diphosphate-ribose polymerase (PARP) enzyme is selected from the group consisting of: olaparib, niraparib, rucaparib, and talazoparib.

40. The method of claim 21, wherein the homologous recombination deficient directed therapy comprises administering a medicament selected from the group consisting of: olaparib, niraparib, rucaparib, and talazoparib.

41. A method of treating a subject that has, or is suspected of having, cancer, the method comprising: a) identifying and/or selecting a subject comprising a genome with a fusion, wherein the fusion comprises at least a portion of a homologous recombinant repair gene; and b) treating the subject so identified and/or selected by administration of an inhibitor of a polyadenosine diphosphate-ribose polymerase (PARP) enzyme or a platinum-based therapy,
wherein identifying and/or selecting a subject comprising a genome with a fusion comprises: i) performing a nucleic acid analysis that preserves spatial-proximal contiguity information on a sample obtained from a subject; and ii) detecting whether the fusion is present or absent in the sample according to the analysis in i).

42. The method of claim 38, wherein the inhibitor of a polyadenosine diphosphate-ribose polymerase (PARP) enzyme is selected from the group consisting of: olaparib, niraparib, rucaparib, and talazoparib.

43. The method of claim 38, wherein the homologous recombinant repair gene is RAD51B.
